(19) United States
(12) Patent Application Publication
(10) Pub. No.: US 2009/0124557 A1
(43) Pub. Date: May 14, 2009

(54) Compositions and methods for treating cancer

(75) Inventors: Nathalie Moyal-Amsellem, Rechovot (IL); Ruth Arnon, Rechovot (IL)

Correspondence Address: MARTIN D. MOYNIHAN d/b/a PRTSI, INC., P.O. BOX 16446, ARLINGTON, VA, 22215, US

(73) Assignee: Yeda Research And Development Co. Ltd., Rechovot (IL)

(21) Appl. No.: 12/081,482

(22) Filed: Apr. 16, 2008

Related U.S. Application Data
(60) Provisional application No. 60/907,766, filed on Apr. 16, 2007.

(51) Int. Cl. A61K 38/08; C12N 1/21

(52) U.S. Cl. 514/16; 435/252.3

(57) ABSTRACT

A composition which comprises a chimeric polypeptide is provided. The chimeric polypeptide having a flagellin amino acid sequence and a mucin 1 amino acid sequence which includes at least a 7 amino acid sequence of the mucin 1 tandem repeat which can be used to elicit an immune response against MUC1—expressing cancerous cells. Also provided is a method of treating cancer such as a cancer of a glandular epithelium in which MUC1 is overexpressed using the composition of the present invention.

### RELATED APPLICATIONS

This is a U.S. patent application which claims priority from U.S. Provisional Patent Application No. 60/907,766, filed on Apr. 16, 2007. The contents of the above-mentioned application are incorporated herein by reference.

### FIELD AND BACKGROUND OF THE INVENTION

The present invention relates to compositions which can be used to elicit a cancer-associated MUC1—specific immune response, and more specifically, to methods and pharmaceutical compositions using same for treating cancer.

According to the world health organization (WHO), more than 11 millions people are diagnosed with cancer every year in the world. Conventional therapies for cancer involve the administration of anti-tumor drugs such as thymidylate synthase inhibitors (e.g., 5-fluorouracil), nucleoside analogs [e.g., gemcitabine (Gemzar)], non-steroidal (e.g., anastrozole and letrozole) and steroidal (exemestane) aromatase inhibitors, taxanes and topoisomerase-I inhibitors (e.g., irinotecan). However, the use of such drugs often fails due to the development of drug resistance by the cancer cells. In addition, in spite of using common anti-cancer treatment modalities, cancer causes 7 millions deaths every year. There is thus, a long felt need, to develop new approaches for preventing and/or treating cancer.

An anti-tumor vaccine is an attractive approach for preventing and/or treating cancer. Anti-tumor vaccines were developed against various tumor-specific antigens. For example, a DNA vaccine based on a shuffled E7 oncogene of the human papilloma virus type 16 (HPV 16) was tested on animals and induced E7-specific cytotoxic T cells (Osen W., et al., 2001, Vaccine, 19: 4276-86). Moreover, a recent study describes the results of a phase I/II trial of a WT1 (Wilm's tumor gene) peptide vaccine (HLA-A*2402-restricted modified 9-mer WT1 peptide) in patients with solid malignancy (Morita S, et al., Jpn J. Clin. Oncol. 2006, 36:231-6).

Mucin 1 (MUC1) is a large transmembrane molecule (>200 kDa), of which the extracellular domain is highly glycosylated (>50%), and contains a tandem repeat (TR) of 20 amino acids which is repeated 60 to 100 times depending on the allele. MUC1 is expressed on the apical surface of most of the glandular epithelial cells. Thus, MUC1 is found in mammary glands (acini and ducts), salivary glands (ducts), esophageal epithelium, stomach (lining and chief cells), pancreas (acini and ducts), bile ducts, lung epithelium, kidney (distal tubules and collecting ducts), bladder (urothelium), uterus (endometrium), rete testis and lymphocytes. In malignant cells, MUC1 is overexpressed, redistributed over the full surface of the cell and its glycosylation pattern is altered, exposing new epitopes on the core protein. Consequently, the MUC1 expressed on malignant cells is antigenically distinct from the MUC1 expressed on normal cells. Moreover, studies utilizing animal models such as transgenic mice and chimpanzees demonstrated that the immune system can differentially destroy MUC1—expressing tumor cells and not MUC1—expressing normal cells (Finn O J. 2003). Therefore, MUC1 is a candidate of choice for immunotherapy of many carcinomas such as breast cancer, lung cancer, gastric cancer and pancreatic cancer. In addition, since MUC1 expression level correlates with the aggressiveness of the tumor and is consequently associated with poorer prognosis, MUC1 is a target for therapy in non-advanced as well as in advanced disease, which are more refractory to existing treatment (Finn O J. 2003). As a result, it is highly desirable to develop an epitope-based anti-tumor vaccine directed against MUC1.

The flagella carrier system has been designed to activate immune responses to linear epitopes genetically fused to flagellin, the structural subunit of the flagella filament. Immunization with the recombinant flagella expressing epitopes of various sequences such as viral, bacterial pathogens and parasites was shown to evoke humoral as well as cellular immune responses against the inserted epitope, which resulted in protection against a challenge infection. The approach of epitope-based vaccines was studied extensively mainly towards the development of an influenza vaccine (Levi R, 1996). Additionally, the present inventors have demonstrated that the flagella do not present a carrier suppression effect on the immune response against the inserted epitope. Finally, it is well known that Flagella is a ligand for a receptor (Toll like receptor 5) belonging to the family of toll like receptors (TLR) which links innate and adaptive immunity. The interaction of TLR with their ligands leads to pro-inflammatory cytokines secretion, to the maturation of dendritic cells, and suppresses the effect of CD4+CD25+ regulatory T cells. Consequently, TLR engagement generally appears to favor Th1 response. Altogether, the flagella present an attractive carrier of foreign sequences for generating an immune response.

In light of its strong immunostimulatory properties, flagellin, the monomeric unit in the flagella, was suggested as an adjuvant for an anti-cancer vaccine. Sotomayor E M., et al. (U.S. Patent Application Publication No. 2006/0088555) describe an anti-tumor vaccine using a flagellin-expressing, lethally irradiated cells which are co-administered with lethally irradiated tumor cells presenting tumor associated antigen. However, administration of tumor cells, even following exposure to lethal irradiation, is unsafe and may result in cancer progression.

The present inventors have previously suggested the use of a chimeric flagellin composed of a flagellin protein and endogenous sequences-of-interest, such as tumor-associated antigens (e.g., mucin 1) in order to generate an anti-cancer prophylactic and/or therapeutic vaccine (Nathalie Moyal-Amsellem, et al., Abstract, Conference p103, Inaugural Joint American-Israeli Conference on Cancer, Novel Therapeutic Approaches to Cancer, 2005). However, to date optimization of a chimeric flagellin with selected mucine-1—epitope sequences for treating cancer has not been shown or taught.

There is thus a widely recognized need for, and it would be highly advantageous to have, a method of treating cancer using a flagella-based anti-cancer vaccine devoid of the above limitations.

### SUMMARY OF THE INVENTION

According to one aspect of the present invention there is provided a method of treating cancer in a subject in need thereof, the method comprising administering to the subject a therapeutically effective amount of a composition which comprises a polypeptide, the polypeptide having an amino acid sequence of a flagellin and an amino acid sequence of a mucin 1, the amino acid sequence of the mucin 1 comprises at least a 7 amino acid sequence of SEQ ID NO:6, thereby treating the cancer in the subject.

According to another aspect of the present invention there is provided a composition-of-matter comprising a polypeptide, the polypeptide having an amino acid sequence of a flagellin and an amino acid sequence of a mucin 1, the amino acid sequence of the mucin 1 comprises at least a 7 amino acid sequence of SEQ ID NO:6.

According to yet another aspect of the present invention there is provided a bacterial host cell being transformed with a nucleic acid construct encoding a polypeptide having an amino acid sequence of a flagellin and an amino acid sequence of a mucin 1, the amino acid sequence of the mucin 1 comprises at least a 7 amino acid sequence of SEQ ID NO:6.

According to still another aspect of the present invention there is provided a pharmaceutical composition comprising a therapeutically effective amount of a composition which comprises a polypeptide, the polypeptide having an amino acid sequence of a flagellin and an amino acid sequence of a mucin 1, the amino acid sequence of the mucin 1 comprises at least a 7 amino acid sequence of SEQ ID NO:6, and a pharmaceutical acceptable carrier.

According to an additional aspect of the present invention there is provided a use of a composition which comprises a polypeptide, the polypeptide having an amino acid sequence of a flagellin and an amino acid sequence of a mucin 1, the amino acid sequence of the mucin 1 comprises at least a 7 amino acid sequence of SEQ ID NO:6, for the manufacture of a medicament identified for treating cancer.

According to further features in preferred embodiments of the invention described below, the amino acid sequence of the flagellin is a contiguous amino acid sequence.

According to still further features in the described preferred embodiments the amino acid sequence of the mucin 1 is positioned at an N-terminal end of the contiguous amino acid sequence of the flagellin.

According to still further features in the described preferred embodiments the amino acid sequence of the mucin 1 is positioned at a C-terminal end of the contiguous amino acid sequence of the flagellin.

According to still further features in the described preferred embodiments the amino acid sequence of the flagellin is a non-contiguous amino acid sequence.

According to still further features in the described preferred embodiments the amino acid sequence of the mucin 1 is flanked by two amino acid segments of the non-contiguous amino acid sequence of the flagellin.

According to still further features in the described preferred embodiments the cells of the cancer express MUC1.

According to still further features in the described preferred embodiments the therapeutically effective amount of the composition is selected capable of eliciting a specific immune response against the cancer in the subject.

According to still further features in the described preferred embodiments the medicament is formulated to elicit an immune response against the cancer.

According to still further features in the described preferred embodiments the immune response is a cellular immune response.

According to still further features in the described preferred embodiments the immune response is a cellular and humoral immune response.

According to still further features in the described preferred embodiments the immune response is capable of inhibiting growth of cells of the cancer.

According to still further features in the described preferred embodiments the amino acid sequence of the mucin 1 is selected from the group consisting of SEQ ID NO:1, 2, 5, 6 and 7.

According to still further features in the described preferred embodiments the cancer affects glandular epithelium.

According to still further features in the described preferred embodiments the cancer is selected from the group consisting of breast cancer, lung cancer, salivary gland cancer, gastric cancer, pancreatic cancer, bile duct cancer, kidney cancer, ovarian cancer, uterus cancer, testis cancer, prostate cancer and bladder cancer.

According to still further features in the described preferred embodiments the cancer is a hematological malignancy.

According to still further features in the described preferred embodiments the hematological malignancy is selected from the group consisting of lymphoma, AML and myeloma.

According to still further features in the described preferred embodiments the cancer is cancer metastases.

According to still further features in the described preferred embodiments the flagellin is a salmonella flagellin.

According to still further features in the described preferred embodiments the polypeptide is substantially pure.

According to still further features in the described preferred embodiments the bacterial host cell is devoid of endogenous flagellin.

The present invention successfully addresses the shortcomings of the presently known configurations by providing a composition capable of eliciting a cancer-associated MUC1 specific immune response which can be used to treat cancer.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a graph depicting the size of tumor (cm³) as a function of time (in days) post implantation of 4T1-MUC1 cells. Balb/c mice were immunized 3 times with adjuvant [first immunization in complete Freund's adjuvant (CFA), and 2 boosts in incomplete Freund's adjuvant (IFA)] at 4 weeks intervals with 100 μg Fla-MUC1.7 (orange triangles; n=9), 100 μg Fla (green squares; n=6) or adjuvant only (blue diamonds; n=7). Four months following the last boost, the mice were implanted with 1.5×10⁶ 4T1-MUC1 cells and tumor growth was monitored. Note that mice immunized with Fla-MUC1.7 present an average tumor size significantly more than 4 times smaller than the average tumor size of the mice immunized with PBS;

FIG. 2 is a histogram depicting the effect of a therapeutic Fla-MUC1.7 vaccine on tumor growth as determined by tumor size (cm³). Balb/c female mice were subcutaneously (s.c.) implanted with 1.5×10⁶ 4T1-MUC1 cells and 10 days post-implantation mice were separated in two group denoted A and B: mice bearing a measurable tumor (A) and mice with palpable but not measurable tumor (B). The two groups of mice were represented in the same proportion in 4 immunized groups which were injected with: 100 μg of Fla in CFA (Fla+CFA; n=8), 100 μg of Fla-MUC1.7 in CFA (Fla-MUC1+CFA; n=8), CFA (control group; n=5) or PBS (control group; n=4). Shown are the sizes of tumors (cm³) as measured in at the first day of immunization (day 0, grey bars) and at day 19 post-immunization (day 19, black bars). Note the significant suppression of tumor size in mice immunized with the Fla-MUC1.7 vaccine;

FIG. 3 is a graph depicting the IgG response (in O.D. units at 450 nm) to MUC1 in Balb/c immunized mice of the experiment described in FIG. 2 hereinabove. The IgG anti-MUC1 was determined using the MUC1 repeated sequence (GVTSAPDTRPAPGSTAPPAH; SEQ ID NO:6) coupled to BSA on ELISA plates as described under “General Materials and Experimental Methods” of the Examples section which follows. Note that at a dilution of 1/125, the IgG anti-MUC1 titer is 3.2 fold higher in mice immunized with Fla-MUC1.7 (orange triangles) than the IgG anti-MUC1 in mice immunized with Fla-CFA (green circles) or mice immunized with CFA (blue squares);

FIG. 4 is a histogram depicting tumor growth (determined by tumor size in cm³) as a function of immunization protocol. Balb/c female mice were implanted s.c. with 1.5×10⁶ 4T1-MUC1 cells and one week post-implantation, the mice were immunized with 100 μg of Fla in CFA (Fla+CFA; n=4), 100 μg of Fla-MUC1.7 in CFA (Fla-MUC1+CFA; n=8), 100 μg of Fla (not shown), 100 μg of Fla-MUC1.7 (Fla-MUC1; n=8), CFA (control group; n=5) or PBS (control group; n=5). Tumor size (cm³) was measured at the first day of immunization (day 0; grey bars) and 30 days post-immunization (day 30; black bars). Because 6 of the 8 mice of the group of mice immunized with Fla alone died 2 days post-immunization, probably due to contamination with endotoxins in the preparation, this group is not represented in the graph. Note the efficient suppression of tumor growth in mice immunized with falgellin-MUC1.7 without adjuvant (e.g., CFA) than in the presence of adjuvant;

FIG. 5 is a graph depicting the IgG response (in O.D. units at 450 nm) to MUC1 in Balb/c immunized mice of the experiment described in FIG. 4 hereinabove. The IgG anti-MUC1 was determined using the MUC1 repeated sequence (GVTSAPDTRPAPGSTAPPAH; SEQ ID NO:6) coupled to BSA on ELISA plates as described under “General Materials and Experimental Methods” of the Examples section which follows. The IgG response anti-MUC1 was assessed in one mouse per group. Note that the mouse immunized with Fla-MUC1.7 presents a very high titer of IgG anti-MUC1 as compared to the mouse from control groups or even as compared to the mouse immunized with Fla-MUC1.7+CFA. Also note that while the mouse immunized with Fla-MUC1.7+CFA does not display higher antibody titer than the mouse from the control groups, the mouse immunized with CFA shows a high antibody titer, as its tumor size is not different from the other control groups;

FIG. 6 is a graph depicting tumor growth as a function of days post immunization with Fla-MUC1-9. Balb/c female mice were implanted s.c. with 1.5×10⁶ 4T1-MUC1 cells. 10 days post-implantation mice were immunized s.c. with 100 μg of Fla-MUC1.9 (FM9; blue triangles; n=15), control Fla-NP (FNP; grey squares; n=8) or control PBS (black squares; n=9) following which tumor size (cm³) was measured at the noted days. Due to differences in the size of tumor at the first day of immunization, the results are presented as a delta of tumor growth (compared to the first day of immunization). Note that 14 days post-immunization mice from the two control groups (FNP and PBS) had to be euthanized due to the size of tumor, whereas the average tumor size of the mice immunized with Fla-MUC1.9 were just reaching back the initial size of the tumor as of the day of immunization;

FIG. 7 is a graph depicting tumor growth as a function of days post immunization with different doses of Fla-MUC1.7 and Fla-MUC1.9. Balb/c female mice (8 weeks old) were implanted s.c. with 1.5×10⁶ 4T1-MUC1 cells and 10 days post-implantation, mice were immunized with 20 μg of Fla-MUC1.7 (FM7 20 μg; n=6), 20 μg of Fla-MUC1.9 (FM9 20 μg; n=6), 50 μg of Fla-MUC1.7 (FM7 50 μg; n=6), 50 μg of Fla-MUC1.9 (FM9 50 μg; n=6), 100 μg of Fla-MUC1.7 (FM7 100 μg; n=6), 100 μg of Fla-MUC1.9 (FM9 100 μg; n=6) or control PBS (n=12) and tumor size was measured (cm³) at the noted days (time post immunization; days). Note that while immunization of mice with 50 or 100 μg/per mouse of either Fla-MUC1.7 or Fla-MUC1.9 resulted in a significant inhibition of tumor growth as compared to PBS control, immunization of mice with 20 μg/per mouse of Fla-MUC1.7 per mouse was not sufficient in order to slow down tumor growth. On the other hand, immunization of mice with 20 μg of Fla-MUC1.9 resulted in efficient inhibition of tumor growth;

FIG. 8 is a graph depicting tumor growth (measured in cm³) as a function of immunization protocol using co-administration of the two flagellin-MUC1 vaccines of the present invention. Balb/c female mice (8 weeks old) were implanted s.c. with 1.5×10⁶ 4T1-MUC1 cells and 10 days post-implantation mice were immunized with 50 μg of Fla-MUC1.7 (FM7; n=10), 50 μg of Fla-MUC1.9 (FM9; n=10), 50 μg of Fla-MUC1.7+50 μg of Fla-MUC1.9 (FM7+FM9; n=10), 50 μg of Fla-NP (FNP; n=5) or PBS control (n=10) and tumor size was measured (cm³) at the noted days. Note that the combination of the two vaccines Fla-MUC1.7 and Fla-MUC1.9 improved the inhibitory effect on tumor growth as compared to when each vaccine was administered alone;

FIG. 9 is a graph depicting tumor growth (measured in cm³) as a function of immunization protocol using multiple immunizations. Balb/c female mice (8 weeks old) were implanted s.c. with 1.5×10⁶ 4T1-MUC1 cells and immunizations were performed with the same dose at 10, 20 and 27 days post implantation. Immunization dose included 50 μg of Fla-MUC1.7 (FM7; n=12), 50 μg of Fla-MUC1.9 (FM9; n=12), 50 μg of Fla-MUC1.7+50 μg of Fla-MUC1.9 (FM7+FM9; n=12) or PBS (control; n=10). Note that slowing down of the tumor growth was achieved by the first and the second immunization with FM7, FM9 and FM7+FM9 whereas the third immunization had no significant effect;

FIG. 10 is a breeding scheme of MUC1 transgenic mice with Balb/c mice in order to obtain generations of MUC1 transgenic mice (F1 and F2 minimum) that could potentially accept the 4T1-MUC1 cell line (which is growing in Babl/c);

FIG. 11 is a graph depicting tumor growth (in %) in human MUC1 transgenic mice which were immunized with the Fla-MUC1.7 vaccine. The experiment was carried out with the F2 generation obtained by successive crossings as illustrated in FIG. 10. F2 mice (12 months old) were implanted s.c. with 1.5×10⁶ 4T1-MUC1 cells and 10 days post-implantation mice were immunized with 100 μg of Fla-MUC1.7 (Fla-MUC1; n=5) or 100 μg Fla-NP (Fla-NP; N=5 control group). Due to differences of tumor size between the 2 groups (group 2 presented higher tumor average comparing to group 1) at day 0 (day of immunization), normalization was applied. Shown is the ratio between the tumor size at different time points and the tumor size at day 0. Note that at day 16, the group of mice immunized with Fla-MUC1.7 presents an average tumor size which is significantly smaller (by 4 times, p<0.01) than the average tumor size in the group immunized with the flagellin carrying a non relevant epitope (Fla-NP);

FIG. 12 is a graph depicting the IgG response (in O.D. units at 450 nm) anti-MUC1 in human MUC1 transgenic mice immunized as described in FIG. 11 hereinabove. Note that the transgenic mouse immunized with Fla-MUC1.7 exhibits much less antibody than the control mice;

FIGS. 13a-b are graphs depicting the IgG response (in O.D. units at 450 nm) anti-MUC1 in Balb/c mice (not implanted with tumor cells) immunized 3 (FIG. 13a) or 4 (FIG. 13b) times with the flagellin-MUC1 vaccines of the present invention. Mice were immunized in 4 weeks intervals, with 100 μg of different recombinant flagellins (Fla-MUC1-7, Fla-NP or PBS) with adjuvant (Adj.) (first immunization in CFA and other boosts in IFA) and sacrificed 8 to 10 days after the last immunization. Serum obtained from the mice was diluted from 1/3 to 1/6661 and was further subjected to IgG response assays. Note that the antibody titer was quite similar in all mice immunized with the different recombinant flagellin proteins.

FIGS. 14a-b are histograms depicting in vitro proliferation assays in the presence of killed 4T1-MUC1 cells (FIG. 14a) or the flagella (FIG. 14b) in mice not implanted with tumor cells. Balb/c mice were immunized 4 times, in 4 weeks intervals, with 100 μg of the flagellin-MUC1-7, the Fla-NP or PBS [all injections, including the PBS control, included adjuvant (first immunization in CFA and other boosts in IFA)]. Mice were sacrificed 8 to 10 days after the last immunization, spleens were removed from the mice and proliferation assay was performed [results are shown in CPM units (left) and fold activation (right)]. FIG. 14a—Note the reactivity of splenocytes from mice immunized with Fla-MUC1-7 to killed 4T1-MUC1 cells; FIG. 14b-Note the reactivity to the flagella of splenocytes from mice immunized with Fla-MUC1.7 or Fla-NP;

FIGS. 15a-b are sequence diagrams depicting part of the sequence of the flagellin gene flanking the insertion site used to ligate the selected MUC1 epitopes. FIG. 15a—Shown is SEQ ID NO:17 which includes the EcoRV restriction site (nucleotides 349-354, shown in bold) which was used to insert the coding sequences encoding the MUC1 epitopes: MUC1.7 (SEQ ID NO:1) or MUC1.9 (SEQ ID:2). The arrow points at the digestion site of EcoRV; FIG. 15b—Shown is SEQ ID NO:28 which includes both AgeI restriction site (nucleotides 313-318, shown in bold) and the EcoRV restriction site (nucleotides 349-354, shown in bold) which was used to insert the coding sequences encoding the MUC1 epitopes: MUC1.20 (SEQ ID NO:6), MUC1.22 (SEQ ID NO:7) or MUC1.25 (SEQ ID NO:5). The arrows point at the digestion sites of EcoRV and AgeI. Note that in order to create the AgeI restriction site in the flagellin vector, the pLS408 plasmid was modified such that the AATGGT nucleic acid sequence (nucleotides 313-318 of SEQ ID NO:17) was replaced by the ACCGGT nucleic acid sequence (nucleotides 313-318 of SEQ ID NO:28; modified nucleotides are underlined);

FIG. 16 is a graph depicting tumor growth (measured in cm³) as a function of immunization protocol using the flagellin-MUC1.25 (MUC1.25 peptide is set forth by SEQ ID NO:5) and the co-administration of the two flagellin-MUC1 vaccines carrying the two epitopes MUC1.7 (SEQ ID NO:1) and MUC1.9 (SEQ ID NO:2) of the present invention. Balb/c female mice (8 weeks old) were implanted s.c. with 1.5×10⁶ 4T1-MUC1 cells and 10 and 23 days post-implantation mice were immunized with 100 μg of Fla-MUC1-25 (FM25; n=10), 50 μg of Fla-MUC1.7+50 μg of Fla-MUC1.9 (FM7+FM9; n=10) or PBS control (n=8) and tumor size was measured (cm³) at the noted days following the first immunization (day 10 following implantation with the 4T1-MUC1 cells). Note that until 10 days after the immunization, the average tumor size of the mice immunized with Fla-MUC1.25 was smaller than the tumor on the first day of immunization. Moreover, the second immunization, which was performed 13 days after the first one (on day 23 post implantation with the 4T1-MUC1 cells, indicated with an arrow) slowed down tumor growth in both immunized group with Fla-MUC1.25 and Fla-MUC1-7+Fla-MUC1.9;

FIG. 17 is a graph displaying tumor growth (measured in cm³) in human MUC1 transgenic mice, which were immunized with the Fla-MUC1.7+Fla-MUC1.9 vaccines. The experiment was carried out with mice from the F1 generation obtained by crossing MUC1 transgenic mice of C57Black background with Balb/c mice. F1 female mice (8-12 weeks old) were implanted s.c. with 1×10⁶ 4T1-MUC1 cells followed by immunization with 50 μg of Fla-MUC1-7+50 μg of Fla-MUC1.9 (n=4) or PBS (control group; n=3) at 10 and 23 days post-implantation. Note that the combination of both vaccines inhibited tumor growth. Thus, at 27 days post implantation mice immunized with the combination of both vaccines present a tumor growth which is more than twice smaller than in the control group;

FIG. 18 is a graph depicting the IgG3 response (in O.D. units at 450 nm) anti-MUC1 in Balb/c mice bearing 4T1-MUC1 tumor immunized twice (10 and 20 days post-implantation) with the flagellin-MUC1 vaccines of the present invention. Mice were implanted s.c. with 1×10⁶ 4T1-MUC1 cells followed by immunization with 50 μg of Fla-MUC1.7, 50 μg of Fla-MUC1-9, 50 μg of Fla-MUC1.7+50 μg of Fla-MUC1.9, 50 μg of Fla-NP or PBS (control group) 10 and 20 days post-implantation. 37 days post implantation mice were sacrificed and serum was collected. To assess IgG3 antibody, ELISA using the peptide set forth by SEQ ID NO:6 not coupled to BSA was performed on serum diluted 1:10 as described in the “General Materials and Experimental Methods”. Note that IgG3 titer is at least 3 times higher in mice immunized with Fla-MUC1.7, or Fla-MUC1.9 or with Fla-MUC1.7+Fla-MUC1.9 as compared with the control group immunized with Fla-NP or PBS;

FIG. 19 is a graph depicting tumor growth (cm³) as a function of immunization protocol using the flagellin-MUC1.25 (MUC1.25 peptide is set forth by SEQ ID NO:5) and the co-administration of the two flagellin-MUC1 vaccines carrying the two epitopes MUC1.7 (SEQ ID NO:1) and MUC1.9 (SEQ ID NO:2) of the present invention in human MUC1 transgenic mice. The experiment was carried out with mice from the F8 generation obtained by successively crossing MUC1 transgenic mice of C57Black background with Balb/c mice. Such mice are considered to be a new strain of human MUC1 transgenic mice on Balb/c genetic Background. Fifteen female mice (8 weeks old) were implanted s.c. with 1.5×10⁶ 4T1-MUC1 cells. 13 days post-implantation the mice were immunized with 50 μg of Fla-MUC1.7+50 μg of Fla-MUC1.9 or 100 μg Fla-MUC1-25 (n=5) or PBS (control group; n=5) at. Note that more than 25 days post-immunization, the average tumor growth of the mice immunized with Fla-MUC1.7+Fla-MUC1.9 (FM7+FM9; n=5) was twice slower than the one of the control group. Moreover, mice immunized with Fla-MUC1.25 (FM25) exhibit a significant arrest of tumor growth. Thus, tumor growth in this group (FM25) was 4 times slower than in the control PBS treated mice; and

FIG. 20 is a histogram depicting the number of lung metastasis 54 days post-implantation, in human MUC1 transgenic mice (F8) bearing 4T1-MUC1 tumor and immunized 13 days post-implantation with Fla-MUC1.7+Fla-MUC1.9 (FM7+FM9) or Fla-MUC1.25 (FM25) or PBS (control group). Lung metastases are presented in absolute numbers. Note that mice immunized with Fla-MUC1-7+Fla-MUC1.9 or Fla-MUC1.25 have twice less metastasis to the lung as compared as the control group.

### DESCRIPTION OF THE PREFERRED EMBODIMENTS

The present invention relates to compositions which can be used to elicit a cancer-associated MUC1—specific immune response in a subject. Specifically, the present invention is of flagellin-MUC1 fusion polypeptides and of methods and pharmaceutical compositions containing same which can be used to treat cancer in the subject.

The principles and operation of the method of treating cancer according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Conventional cancer therapy using agents such as thymidylate synthase inhibitors, nucleoside analogs, aromatase inhibitors, taxanes and topoisomerase-I inhibitors has limited efficiency. Other treatment regimens using an anti-cancer vaccine have been also explored (Osen W., et al., 2001, Vaccine, 19: 4276-86; Morita S, et al., 2006, Jpn J. Clin. Oncol. 36:231-6).

The altered pattern of expression of MUC1 in cancerous cells promoted the development of an anti-MUC1 vaccine for the prevention and/or treatment of various cancers which are associated with MUC1 over—and altered expression.

The flagella carrier system has been designed to activate immune responses to linear epitopes genetically fused to flagellin, the monomeric subunit of the flagella filament. In light of its strong immunostimulatory properties, flagellin was suggested as an adjuvant for an anti-cancer vaccine. Sotomayor E M., et al. (U.S. Pat. Appl. No. 20060088555) describe an anti-tumor vaccine using flagellin-expressing, lethally irradiated cells which are co-administered with lethally irradiated tumor cells presenting tumor associated antigen. However, administration of tumor cells, even following exposure to lethal irradiation, is unsafe and may result in cancer progression.

The present inventors have previously suggested the use of a chimeric flagellin composed of a flagellin protein and endogenous sequences-of-interest, such as tumor-associated antigens (e.g., mucin 1) in order to generate an anti-cancer prophylactic and/or therapeutic vaccine (Nathalie Moyal-Amsellem, et al., Abstract, Conference p103, Inaugural Joint American-Israeli Conference on Cancer, Novel Therapeutic Approaches to Cancer, 2005). However, to date optimization of a chimeric flagellin with selected mucine-1—epitope sequences to treat cancer has not been shown or taught.

While reducing the present invention to practice, the present inventors have uncovered, following laborious experimentations, that MUC1 specific sequences can be conjugated to the flagellin polypeptide to generate a chimeric flagellin-MUC1 polypeptide capable of inducing a cancer associated MUC1—specific immune response, to thereby treat the growth of solid tumors and as such can be used to treat cancer.

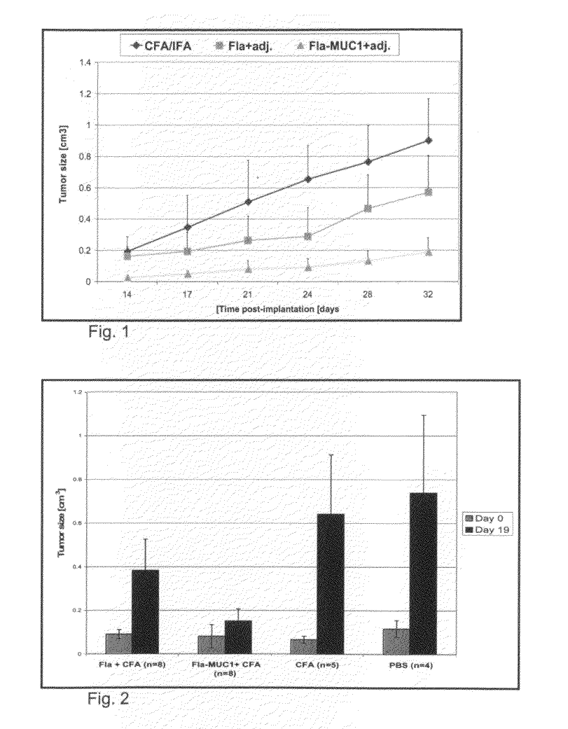

As is shown in FIG. 1 and is described in Example 1 of the Examples section which follows, immunizing mice with the Fla-MUC1.7 polypeptide prior to injection of cancerous cells resulted in a significant protective effect against tumor formation, thus suggesting the use of the Fla-MUC1.7 polypeptide for preventing tumor formation in a subject with increased risk to develop cancer. In addition, as is shown in FIGS. 2, 4, 6, 7, 8, 9, 11, 16, 17, 18, 19 and 20 and is described in Examples 2, 3, 4, 5, 6, 8, 9 and 10 of the Examples section which follows, the Fla-MUC1.7, Fla-MUC1.9 and/or Fla-MUC1.25 chimeric polypeptides were shown capable of inhibiting the growth of existing tumors. These results suggest the use of the Fla-MUC1.7, Fla-MUC1.9 and/or Fla-MUC1.25 polypeptide for treating cancer.

Thus, according to one aspect of the present invention there is provided a composition-of-matter which comprises a polypeptide or a polymer thereof, the polypeptide having an amino acid sequence of a flagellin and an amino acid sequence of a mucin 1, the amino acid sequence of the mucin 1 comprises at least a 7 amino acid sequence of SEQ ID NO:6.

As used herein the phrase “amino acid sequence of a flagellin” refers to an amino acid sequence of a flagellin protein, the main polypeptide monomer of the bacterial flagella, which is capable of eliciting an immune response (used as an adjuvant) in a subject. Preferably, the amino acid sequence of the flagellin used by the present invention is selected such that it can be attached to a mucin 1 amino acid sequence (as is further described hereinbelow) and yet preserves its function as an adjuvant.

The amino acid sequence of the flagellin used in the composition-of-matter of this aspect of the present invention can be derived from any bacterial flagella such as Escherichia, Salmonella, Proteus, Pseudomonas, Bacillus, Campylobacter, Vibrio, Treponema, Legionella, Clostridia and Caulobacter spp. Preferably, the flagellin amino acid sequence used in the composition-of-matter of this aspect of the present invention is derived from Salmonella Munchen flagella [GenBank Accession No. X03395 (SEQ ID NO:32; nucleic acid sequence); PO6177 (SEQ ID NO:11; amino acid sequence). For example, as shown in FIGS. 15a-b and described in the Examples section which follows, a vector containing the flagellin nucleic acid sequence (which comprises SEQ ID NO:17 or 28), which encodes a flagellin amino acid sequence (comprising SEQ ID NO:10 or 31), was used for ligation of a mucin 1 amino acid sequence therein.

As mentioned, the above-described amino acid sequence of flagellin can be attached (e.g., covalently bound e.g., via a peptide bond or conjugation via a linker, so as to form a chimeric/fusion polypeptide) to an amino acid sequence of mucin 1 which comprises at least a 7 amino acid sequence of SEQ ID NO:6.

As used herein the term “mucin 1” refers to the amino acid sequence of the cell surface associated mucin 1 polypeptide [MUC1; GenBank Accession No. P15941 (SEQ ID NO:12)], a large transmembrane molecule (>200 kDa), of which the extracellular domain is highly glycosylated (≧50%), and which contains a tandem repeat (TR) of 20 amino acids (as set forth in SEQ ID NO: 6) which is repeated 60 to 100 times depending on the allele. As mentioned in the background section, in most of the normal glandular epithelial cells MUC1 is expressed on the apical surface. However, in malignant cells, MUC1 is overexpressed, redistributed over the full surface of the cell and its glycosylation pattern is altered thus exposing new epitopes on the core protein. Consequently, the MUC1 expressed on malignant cells is antigenically distinct from the MUC1 expressed on normal cells.

Preferably, the mucin 1 amino acid sequence which is included in the polypeptide of this aspect of the present invention is a mucin 1 epitope used to elicit a cancer associated MUC1 specific immune response in the subject.

As used herein, the term “epitope” refers to any antigenic determinant of an antigen to which the paratope of an antibody binds. As used herein the phrase “cancer associated MUC1 specific immune response” refers to an immune response which is specific against MUC1 as expressed on a cancerous cell. Preferably, the mucin 1 amino acid sequence which forms a part of the polypeptide of the present invention includes at least a 7 amino acid sequence (i.e., 7 consecutive amino acids) of SEQ ID NO:6. It will be appreciated that the mucin 1 amino acid sequence of the present invention can be any 7 consecutive amino acids of the amino acid sequence set forth by SEQ ID NO:6.

Preferably, the mucin 1 amino acid sequence which is included in the polypeptide of the present invention may include at least a 7 amino acid sequence, at least an 8 amino acid sequence, at least a 9 amino acid sequence, at least a 10 amino acid sequence, at least an 11 amino acid sequence, at least a 12 amino acid sequence, at least a 13 amino acid sequence, at least a 14 amino acid sequence, at least a 15 amino acid sequence, at least a 16 amino acid sequence, at least a 17 amino acid sequence, at least an 18 amino acid sequence, at least a 19 amino acid sequence or at least a 20 amino acid sequence of the amino acid sequence set forth by SEQ ID NO:6. Non-limiting examples of the mucin 1 amino acid sequence which is included in the polypeptide of the present invention include the amino acid sequences set forth by SEQ ID NOs:1 and 2.

It will be appreciated that the mucin 1 amino acid sequence which is included in the polypeptide of the present invention can include at least a 7 amino acid sequence of SEQ ID NO:6 and additional amino acid(s) which are derived from the same repeat unit, from the proceeding repeat unit and/or from the subsequent repeat unit. For example, the mucin 1 amino acid sequence which is included in the polypeptide of the present invention can be a 9 amino acid sequence as set forth by SEQ ID NO:13 which includes amino acids 19-20 of SEQ ID NO:6 followed by amino acids 1-7 of SEQ ID NO:6. Additionally or alternatively, the mucin 1 amino acid sequence which is included in the polypeptide of the present invention can be a 9 amino acid sequence as set forth by SEQ ID NO:14 which includes amino acids 14-20 of SEQ ID NO:6 followed by amino acids 1-2 of SEQ ID NO:6.

Still additionally or alternatively, the mucin 1 amino acid sequence which is included in the polypeptide of the present invention can include more than 20 amino acids in length, such a mucin 1 amino acid sequence can include the 20 amino acid sequence set forth by SEQ ID NO:6 and additional amino acid(s) (e.g., one, two, three, four, five, six, at least seven, at least eight amino acids) which are derived from SEQ ID NO:6. The additional amino acid(s) can be positioned at the N-terminal or the C-terminal end of the amino acid sequence of SEQ ID NO:6. Thus, a mucin 1 amino acid sequence which is included in the polypeptide of the present invention can include at least 21 amino acids, at least 22 amino acids, at least 23 amino acids, at least 24 amino acids, at least 25 amino acids in length which include the amino acid sequence of SEQ ID NO:6 and an additional amino acid sequence which is derived from SEQ ID NO:6 and is positioned at the N-terminal and/or C-terminal of SEQ ID NO:6. Non-limiting examples of such a mucin 1 amino acid sequence is set forth by SEQ ID NOs:5 and 7 and is described in Example 9 of the Examples section which follows.

The polypeptide of the present invention (hereinafter the chimeric polypeptide of the present invention) which includes the above-described amino acid sequence of the flagellin and the above-described amino acid sequence of the mucin 1 which comprises at least the 7 amino acid sequence of SEQ ID NO:6 can be chemically synthesized or recombinantly expressed as is further described hereinunder. It will be appreciated that if the chimeric polypeptide is recombinantly expressed, the nucleic acid sequences which encode the flagellin and mucin 1 amino acid sequences are translationally fused to form a single continuous open reading frame spanning the length of the coding sequences of the linked nucleic acid sequences.

It will be appreciated that the flagellin amino acid sequence included in the chimeric polypeptide of the present invention can be a contiguous amino acid sequence or a non-contiguous amino acid sequence. As used herein the phrase “contiguous amino acid sequence” refers to a continuous amino acid sequence of the flagellin which is not interrupted in its sequence by other amino acid(s) which are derived from other polypeptides such as a mucin 1 polypeptide. Alternatively, the phrase “non-contiguous amino acid sequence” refers to an amino acid sequence of the flagellin which is non-continuous (e.g., interrupted) due to the presence of amino acid(s) derived from other polypeptides such as a mucin 1 polypeptide.

In case a contiguous amino acid sequence of the flagellin is used, the at least 7 amino acid sequence of mucin 1 can be positioned (e.g., placed, present) at the N-terminal or the C-terminal end of the contiguous amino acid sequence of the flagellin. Non-limiting examples of such a polypeptide comprise SEQ ID NO:15 (for a 7 amino acid sequence of mucin 1 which is positioned at the N-terminal end of flagellin) and SEQ ID NO:16 (for a 7 amino acid sequence of mucin 1 which is positioned at the C-terminal end of flagellin).

Alternatively, in case a non-contiguous amino acid sequence of the flagellin is used, the at least 7 amino acid sequence of mucin 1 is flanked by (i.e., is positioned between) the two amino acid segments of the non-contiguous amino acid sequence of the flagellin (i.e., the at least 7 amino acid sequence of mucin 1 forms an integral part within the flagellin amino acid sequence). The position of the at least 7 amino acid sequence of mucin 1 within the amino acid sequence of flagellin is selected such that the chimeric polypeptide of the present invention (which includes a portion of the mucin 1 amino acid sequence within the flagellin amino acid sequence) maintains the function of the non-chimeric flagellin polypeptide (e.g., as a carrier of an antigen, adjuvant for the immune response, enables the salmonella expressing the recombinant flagella to rotate) and those of skills in the art are capable of selecting the appropriate insertion site within the flagellin amino acid sequence. For example, the present inventors have used the expression vectors pls408 (see partial sequence in FIGS. 15a-b) which includes the nucleic acid sequence encoding the flagellin polypeptide with suitable cloning sites for inserting a heterologous nucleic acid sequence encoding an amino acid sequence-of-choice such as the mucin 1 amino acid sequence described hereinabove (which includes at least 7 amino acid sequence of SEQ ID NO:6). Thus, for inserting the MUC1.7 or MUC1.9 nucleic acid sequences the EcoRV restriction site shown in FIG. 15a was utilized. For inserting the MUC1.20, MUC1.22 or MUC1.25 nucleic acid sequences the AgeI and EcoRV restriction sites shown in FIG. 15b were utilized.

It will be appreciated that the chimeric polypeptide of the present invention can be qualified by following the size of tumor in animal models injected with MUC1-expressing cancerous cells, essentially as described in the Examples section which follows.

As used herein, the phrases “polypeptide”, “peptide” or “amino acid sequence” which are interchangeably used herein, encompass a naturally occurring polypeptide which is comprised solely of natural amino acid residues, peptide analogues or mimetics thereof. Further description of natural and non-natural amino acids is provided in PCT Application No. PCT/IL2004/000744, which is fully incorporated herein by reference.

As used herein the term “mimetics” refers to molecular structures, which serve as substitutes for the peptide of the present invention in performing the biological activity (Morgan et al. (1989) Ann. Reports Med. Chem. 24:243-252 for a review of peptide mimetics). Peptide mimetics, as used herein, include synthetic structures (known and yet unknown), which may or may not contain amino acids and/or peptide bonds, but retain the structural and functional features of the peptide. Types of amino acids which can be utilized to generate mimetics are further described hereinbelow. The term, “peptide mimetics” also includes peptoids and oligopeptoids, which are peptides or oligomers of N-substituted amino acids [Simon et al. (1972) Proc. Natl. Acad. Sci. USA 89:9367-9371]. Further included as peptide mimetics are peptide libraries, which are collections of peptides designed to be of a given amino acid length and representing all conceivable sequences of amino acids corresponding thereto. Methods of producing peptide mimetics are described hereinbelow.

As mentioned before, the chimeric polypeptide of the present invention can be chemically synthesized such as by using standard solid phase techniques. These methods include exclusive solid phase synthesis, partial solid phase synthesis methods, fragment condensation and classical solution synthesis. These methods are preferably used when the polypeptide is relatively short (i.e., a 10 kDa peptide) and/or when it cannot be produced by recombinant techniques (i.e., not encoded by a nucleic acid sequence) and therefore involves different chemistry.

Solid phase peptide synthesis procedures are well known in the art and further described by John Morrow Stewart and Janis Dillaha Young, Solid Phase Peptide Syntheses (2nd Ed., Pierce Chemical Company, 1984).

Synthetic peptides can be purified by preparative high performance liquid chromatography [Creighton T. (1983) Proteins, structures and molecular principles. WH Freeman and Co. N.Y.] and the composition of which can be confirmed via amino acid sequencing.

It will be appreciated that for large polypeptides (e.g., above 25 amino acids), the chimeric polypeptide of the present invention is preferably prepared using recombinant techniques.

For example, to generate the chimeric polypeptide of the present invention, a polynucleotide sequence comprising SEQ ID NO:18 or 19 which encodes a flagellin amino acid sequence (e.g., an amino acid sequence comprising SEQ ID NO:10) and the mucin 1 amino acid sequence which comprises at least a 7 amino acid sequence of SEQ ID NO:6 (e.g., the amino acid sequence set forth by SEQ ID NO:1 or 2) is preferably ligated into a nucleic acid construct suitable for expression in a host cell. Such a nucleic acid construct includes a promoter sequence for directing transcription of the polynucleotide sequence in the cell in a constitutive or inducible manner.

The nucleic acid construct (also referred to herein as an “expression vector”) of the present invention includes additional sequences which render this vector suitable for replication and integration in prokaryotes, eukaryotes, or preferably both (e.g., shuttle vectors). In addition, a typical expression vector may also contain a transcription and translation initiation sequence, enhancers (e.g., SV40 early gene enhancer; see also Enhancers and Eukaryotic Expression, Cold Spring Harbor Press, Cold Spring Harbor, N.Y. 1983), transcription and translation terminator, and a polyadenylation signal which may increase the efficiency of mRNA translation (e.g., the GU or U rich sequences located downstream from the polyadenylation site and a highly conserved sequence of six nucleotides, AAUAAA, located 11-30 nucleotides upstream).

The expression vector of the present invention can further include additional polynucleotide sequences that allow, for example, the translation of several proteins from a single mRNA such as an internal ribosome entry site (IRES) and sequences for genomic integration of the promoter-chimeric polypeptide.

Other than containing the necessary elements for the transcription and translation of the inserted coding sequence, the expression construct of the present invention can also include sequences engineered to enhance stability, production, purification, yield or toxicity of the expressed polypeptide.

As mentioned hereinabove, a variety of cells can be used as host-expression systems to express the chimeric polypeptide of the present invention. These include, but are not limited to, microorganisms, such as bacteria transformed with a recombinant bacteriophage DNA, plasmid DNA or cosmid DNA expression vector containing the polypeptide coding sequence. Bacterial systems are preferably used to produce the chimeric polypeptide of the present invention since they enable a high production volume at low cost.

It will be appreciated that in order to generate a flagella-like structure, i.e., a polymer of the flagellin amino acid sequence, the recombinant chimeric polypeptide of the present invention is preferably produced in a bacterial host cell, more preferably, in a bacterial host cell devoid of a flagella [e.g., the flagellin negative live vaccine strain (an Aro A mutant) of S. Dublin SL5928 as described in the “General Materials and Experimental Methods” of the Examples section which follows)].

Various methods can be used to introduce the expression vector of the present invention into host cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Transformed cells are cultured under effective conditions, which allow for the expression of high amounts of the recombinant polypeptide. Effective culture conditions include, but are not limited to, effective media, bioreactor, temperature, pH and oxygen conditions that permit protein production. An effective medium refers to any medium in which a cell is cultured to produce the recombinant polypeptide of the present invention. Such a medium typically includes an aqueous solution having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes and petri plates. Culturing can be carried out at a temperature, pH and oxygen content appropriate for a recombinant cell. Such culturing conditions are within the expertise of one of ordinary skill in the art.

The resultant polypeptides of the present invention are retained on the outer surface of a cell such as a flagella.

Following a predetermined time in culture, recovery of the recombinant polypeptide is effected. The phrase “recovery of the recombinant polypeptide” used herein refers to collecting the whole fermentation medium containing the polypeptide and need not imply additional steps of separation or purification.

Thus, polypeptides of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

Preferably, the polypeptides of the present invention (the chimeric flagella-MUC1) are isolated from the host cells using acid cleavage as described under the “General Materials and Experimental Methods” of the Examples section which follows. Briefly, bacterial host cells expressing the chimeric polypeptide of the present invention are collected from agar plates containing selective agents (e.g., ampicillin) and grown overnight in Luria broth (LB) in the presence of the same selective agents. The bacterial cells are then resuspended in PBS and subjected to acid cleavage in the presence of 6 to 10 N of Chloridric acid (until pH reaches the value of 2). The flagella-containing supernatant from two successive centrifugations (the first one for 15 minutes at 9000 rpm and the second one at 19500 rpm for one hour) is subjected to ammonium sulfate precipitation (35%), centrifugation, resuspension in PBS and dialysis against PBS in the presence of activated charcoal.

Thus, the composition of the present invention which comprises the chimeric polypeptide described hereinabove is retrieved from the recombinant bacterial cells in a “substantially pure” form. As used herein, the phrase “substantially pure” refers to a purity that allows for the effective use of the composition of the present invention in treating cancer.

It will be appreciated that the composition of the present invention can be subjected to a cleaning protocol intended to remove agents such as Lipopolysaccharide (LPS) (a component of the outer membrane of most gram negative microbes). For example, LPS can be removed from the composition of the present invention using an inorganic particulate resin [e.g., a hydrophilic resin such as the commercially available fumed silica product Aerosil™ (Degussa A G, Frankfurt)], which has siloxane and silanol groups on the surface of the particles, essentially as described in U.S. Pat. Appl. No. 20050228171 to More, John Edward et al., which is fully incorporated herein by reference. Aerosil™ and similar resins are in use in purification processes in the pharmaceutical industry for the removal of lipid and lipid-like substances (e.g., LPS) from plasma products such as the plasma glycoprotein Alpha-1-acid glycoprotein (AAG). The purity of the composition can be qualified by measurement of dye binding by LPS [as described in Keler and Nowotny, 1986, Analyt. Biochem., 156:189] or the use of a Limulus amebocyte lysate (LAL) test [see, for instance, Endotoxins and Their Detection With the Limulus Amebocyte Lystate Test, Alan R. Liss, Inc., 150 Fifth Avenue, New York, N.Y. (1982)] such as using the gel-clot test E-TOXATE (Sigma Chemical Co., St. Louis, Mo.; see Sigma Technical Bulletin No. 210) and the turbidimetric (spectrophotometric) test [Sakai H, et al., 2004, J Pharm Sci. 93(2):310-21].

It will be appreciated that since the flagellin polypeptide naturally exists in the bacterial flagella is a polymer composed of several thousands of flagellin monomeric subunits [e.g., about 20,000 subunits (Auvray F, et al., 2001, J. Mol. Biol. 308:221-9)] and since such a flagella is capable of eliciting an immune response in a subject, the composition of the present invention preferably includes a polymer of several monomers of the chimeric polypeptide of the present invention. It will be appreciated that a polymer of the chimeric polypeptide of the present invention can be formed in vitro using cell-free constitutes or in vivo within the bacterial host cells expressing the chimeric polypeptide of the present invention.

For in vitro formation of the polymer of the present invention, the chimeric polypeptide of the present invention can be expressed as monomers in a host cell and be further retrieved from the host cell (e.g., from the cytosolic fraction, the culture medium or the membrane-bound fraction of the host cell) and the isolated monomers can be subject to polymerization in vitro using the cytosolic export chaperon FliS that binds to the C-terminal helical domain of the flagellin-containing chimeric protein monomers (Auvray F, et al., 2001, J. Mol. Biol. 308: 221-9).

Alternatively and currently more preferred, the polymer of the present invention can be formed in vivo in a bacterial host cell (such as the Aro A mutant of S. Dublin SL5928 bacterial cells which are devoid of a flagella) by recombinantly expressing the chimeric polypeptide of the present invention in a bacterial host cell, and the polymer of the recombinant flagella (the chimeric polypeptide of the present invention) is isolated using acid cleavage essentially as described hereinabove, in the Examples section which follows and in Ibrahim et al. Method for the isolation of highly purified Salmonella flagellin. J. Clin. Microbiol; 1985, 22:1040-1044), which is fully incorporated herein by reference.

As mentioned, the present inventors have uncovered, through laborious experimentations, that a chimeric polypeptide such as the Fla-MUC1-7, Fla-MUC1.9 and/or Fla-MUC1.25 which includes at least a 7 amino acid sequence of the MUC1 repeated unit (i.e., the tandem repeat set forth by SEQ ID NO:6) of the mucin 1 polypeptide (SEQ ID NO:12) is sufficient to induce an immune response against mucin 1 and thus treat cancer.

Thus, according to another aspect of the present invention there is provided a method of treating cancer in a subject in need thereof. The method is effected by administering to the subject a therapeutically effective amount of the composition of the present invention described hereinabove, thereby treating the cancer in the subject.

The term “treating” refers to inhibiting, preventing (i.e., keeping from occurring in a subject at risk), curing, reversing, attenuating, alleviating, minimizing, suppressing or halting the deleterious effects of pathology [i.e., non-solid tumor cancer (e.g., hematological malignancy), primary solid tumor cancer and/or cancer metastases of both solid or non-solid origin] and/or causing the reduction, remission, or regression of the pathology. Those of skills in the art will understand that various methodologies and assays can be used to assess the development of the pathology, and similarly, various methodologies and assays may be used to assess the reduction, remission or regression of the pathology.

As used herein, the phrase “subject in need thereof” refers to an animal subject e.g., a mammal, e.g., a human being at any age who suffers from (including a subject who is generally healthy but has been diagnosed with cancer) or is at risk of developing the pathology (e.g., predisposed). Non-limiting examples of subjects who are at risk of developing the pathology of the present invention, include subjects who are genetically predisposed to developing cancer (e.g., subjects who carry a mutation associated with cancer, e.g., a mutation in a gene encoding a tumor suppressor or an oncogene such as in the BRCA1, BRCA2, P53 and/or ATM genes and/or subjects having a family history of cancer), and/or subjects who are at high risk of developing the pathology due to other factors such as environmental hazard (e.g., subjects who are exposed to DNA damaging agents, ionizing radiation and the like).

Preferably, the cancer which may be treated with the composition of the present invention is associated with altered cellular expression pattern of mucin 1.

As used herein the phrase “altered cellular expression pattern of mucin 1” refers to a mucin 1 expression pattern in cancerous (malignant) cells which is mostly different than the one found in normal, unaffected cells (i.e., cells devoid of cancer). For example, while in normal glandular epithelial cells and/or hematological cells MUC1 is expressed on the apical surface, in malignant, cancerous cells, MUC1 is overexpressed and redistributed over the full surface of the cell, its glycosylation pattern is altered, exposing new epitopes on the core protein. Thus, a cancer which can be treated by the method of this aspect of the present invention includes a solid or non-solid primary tumor and/or metastases of both solid origin and non-solid origin (hematopoietic malignancies) which affect cells of the glandular epithelium (i.e., a cancer associated with the glandular epithelium) such as breast cancer, lung cancer, salivary gland cancer, esophageal cancer, gastric cancer, pancreatic cancer, bile duct cancer, kidney cancer, ovarian cancer, uterus cancer, testis cancer, prostate cancer and bladder cancer, colon cancer, or it may be a cancer which affects cells of the hematopoietic system (e.g., lymphocytes) associated with a hematological malignancy such as lymphoma [e.g., systemic anaplastic large cell lymphoma (ALCL), Rassidakis G. Z. et al., 2003, Clinical Cancer Research, 9: 2213-2220], acute myelogenous leukemia (AML) or myeloma (Brossart P, et al., 2001, Cancer Res. 61: 6846-50).

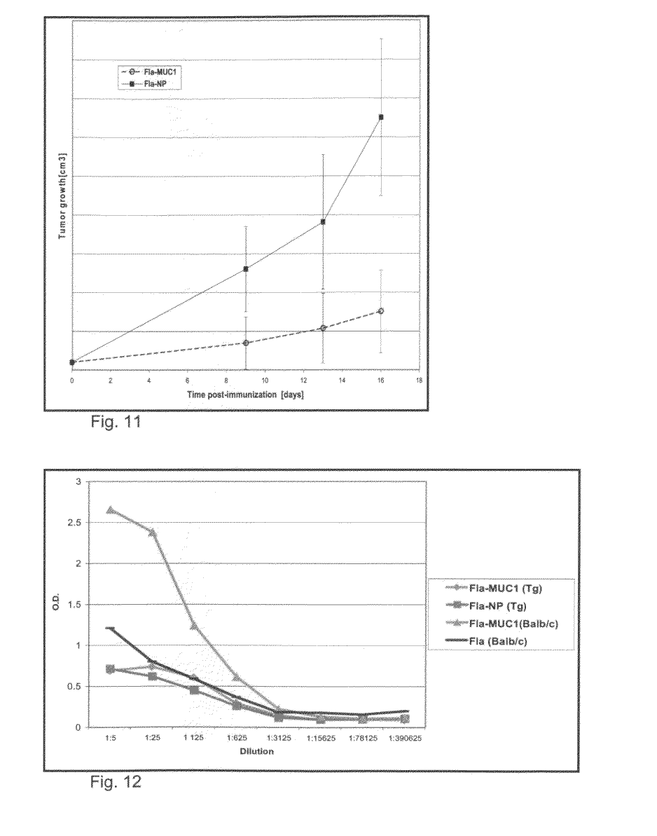

As used herein the phrase “therapeutically effective amount” refers to the amount of the composition of the present invention which is effective to prevent, alleviate or ameliorate symptoms of a pathology or a disorder (the cancer as described hereinabove) or prolong the survival of the subject being treated. Preferably, the therapeutically effective amount of the composition of the present invention is selected capable of eliciting in the subject a MUC1—specific immune response against the cancer. Such an immune response can be a cellular immune response (which involves T lymphocytes) and/or a humoral immune response (which involves B lymphocytes). As is shown in FIGS. 12, 13, 14 and 18 and is described in Example 7 of the Examples section which follows, the immune response elicited by the composition of the present invention seems to be mostly a cellular immune response, which has a strong contribution in preventing the growth of cancerous cells in the subject, in addition to a humoral immune response, which also exhibits a protective effect against tumor growth.

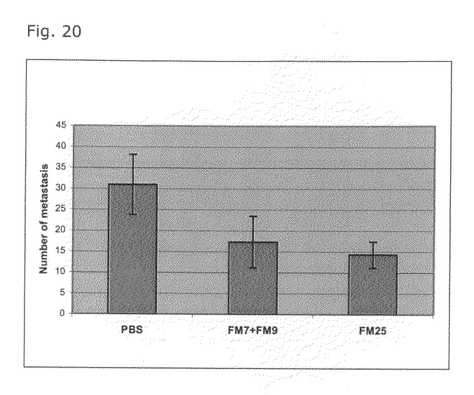

As is mentioned hereinabove and as is shown in FIGS. 2, 4, 6, 7, 8, 9, 11, 16, 17, 18 and 19 and is described in Examples 2, 3, 4, 5, 6, 8, 9 and 10 of the Examples section which follows, the compositions of the present invention such as those including the Fla-MUC1.7 (which comprises SEQ ID NO:8), Fla-MUC1.9 (which comprises SEQ ID NO:9) and/or Fla-MUC1.25 (which comprises SEQ ID NO:22) chimeric polypeptides were shown capable of inhibiting the growth of already established tumors. In addition, as is shown in Tables 2 and 3 and FIG. 20 and is described in Examples 6 and 10 of the Examples section which follows, the compositions of the present invention were capable of reducing the number of cancer metastases. Thus, the immune response elicited by the composition of the present invention is preferably capable of inhibiting the growth of the cancerous cells (e.g., inhibiting and/or slowing down the growth of a solid tumor) and thus capable of treating the cancer. In addition, the immune response elicited by the composition of the present invention is preferably capable of treating cancer metastases.

It will be appreciated that the composition of the present invention which is capable of treating cancer by eliciting an immune response against MUC1—expressing cancerous cells can be administered to an organism per se or in a pharmaceutical composition where it is mixed with suitable carriers or excipients.

As used herein, a “pharmaceutical composition” refers to a preparation of one or more of the active ingredients described herein with other chemical components such as physiologically suitable carriers and excipients. The purpose of a pharmaceutical composition is to facilitate administration of a compound to an organism.

As used herein, the term “active ingredient” refers to the agent accountable for the intended biological effect (i.e., the composition of the present invention).

Hereinafter, the phrases “physiologically acceptable carrier” and “pharmaceutically acceptable carrier,” which may be used interchangeably, refer to a carrier or a diluent that does not cause significant irritation to an organism and does not abrogate the biological activity and properties of the administered compound. In some cases, a physiologically acceptable carrier includes an adjuvant.

Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils, and polyethylene glycols.

Techniques for formulation and administration of drugs may be found in the latest edition of “Remington's Pharmaceutical Sciences,” Mack Publishing Co., Easton, Pa., which is herein fully incorporated by reference.

Suitable routes of administration may, for example, include oral, rectal, transmucosal, especially transnasal, intestinal, or parenteral delivery, including intramuscular, subcutaneous, and intramedullary injections, as well as intrathecal, direct intraventricular, intravenous, inrtaperitoneal, intranasal, or intraocular injections.

Alternately, one may administer the pharmaceutical composition in a local rather than systemic manner, for example, via injection of the pharmaceutical composition directly into a tissue region of a patient.

Pharmaceutical compositions of the present invention may be manufactured by processes well known in the art, e.g., by means of conventional mixing, dissolving, granulating, dragee-making, levigating, emulsifying, encapsulating, entrapping, or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations that can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries as desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, and sodium carbomethylcellulose; and/or physiologically acceptable polymers such as polyvinylpyrrolidone (PVP). If desired, disintegrating agents, such as cross-linked polyvinyl pyrrolidone, agar, or alginic acid or a salt thereof, such as sodium alginate, may be added.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions that can be used orally include push-fit capsules made of gelatin, as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate, and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane, or carbon dioxide. In the case of a pressurized aerosol, the dosage may be determined by providing a valve to deliver a metered amount. Capsules and cartridges of, for example, gelatin for use in a dispenser may be formulated containing a powder mix of the compound and a suitable powder base, such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with, optionally, an added preservative. The compositions may be suspensions, solutions, or emulsions in oily or aqueous vehicles, and may contain formulatory agents such as suspending, stabilizing, and/or dispersing agents.

Pharmaceutical compositions for parenteral administration include aqueous solutions of the active preparation in water-soluble form. Additionally, suspensions of the active ingredients may be prepared as appropriate oily or water-based injection suspensions. Suitable lipophilic solvents or vehicles include fatty oils such as sesame oil, or synthetic fatty acid esters such as ethyl oleate, triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the active ingredients, to allow for the preparation of highly concentrated solutions.

Alternatively, the active ingredient may be in powder form for constitution with a suitable vehicle, e.g., a sterile, pyrogen-free, water-based solution, before use.

The pharmaceutical composition of the present invention may also be formulated in rectal compositions such as suppositories or retention enemas, using, for example, conventional suppository bases such as cocoa butter or other glycerides.

Pharmaceutical compositions suitable for use in the context of the present invention include compositions wherein the active ingredients are contained in an amount effective to achieve the intended purpose.

Determination of a therapeutically effective amount is well within the capability of those skilled in the art, especially in light of the detailed disclosure provided herein.

For any preparation used in the methods of the invention, the dosage or the therapeutically effective amount can be estimated initially from in vivo assays using animal models as described in the Examples section which follows. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration, and dosage can be chosen by the individual physician in view of the patient's condition. (See, e.g., Fingl, E. et al. (1975), “The Pharmacological Basis of Therapeutics,” Ch. 1, p. 1.)

Dosage amount and administration intervals may be adjusted individually to provide sufficient tissue levels (e.g., plasma, brain, lung) of the active ingredient to induce or suppress the biological effect (i.e., minimally effective concentration, MEC). The MEC will vary for each preparation, but can be estimated from in vitro data. Dosages necessary to achieve the MEC will depend on individual characteristics and route of administration. Detection assays can be used to determine plasma concentrations.

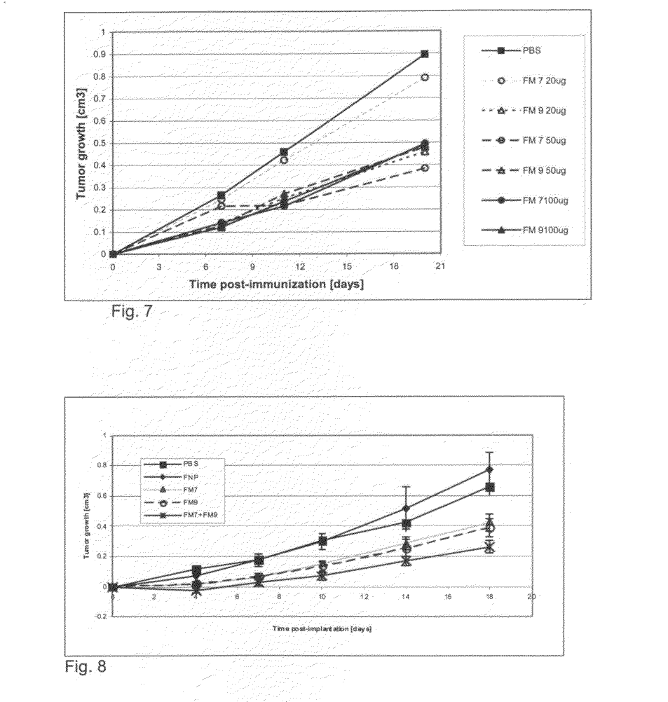

Depending on the severity and responsiveness of the condition to be treated, dosing can be of a single or a plurality of administrations, with course of treatment lasting from several days to several weeks, or until cure is effected or diminution of the disease state is achieved. For example, as is shown in FIG. 7 and is described in Example 4 of the Examples section which follows, a single dose of 50 or 100 μg, but not of a 20 μg of the composition comprising the Fla-MUC1.7 chimeric polypeptide was efficient in inhibiting tumor growth. On the other hand, a single dose of 20, 50 or 100 μg the composition which comprises the Fla-MUC1.9 chimeric polypeptide resulted in efficient inhibition of tumor growth.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA-approved kit, which may contain one or more unit dosage forms containing the active ingredient. The pack may, for example, comprise metal or plastic foil, such as a blister pack. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising a preparation of the invention formulated in a pharmaceutically acceptable carrier may also be prepared, placed in an appropriate container, and labeled for treatment of an indicated condition (e.g., pathology), as further detailed above.

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., Ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (Eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., Ed. (1994); “Culture of Animal Cells—A Manual of Basic Technique” by Freshney, Wiley-Liss, N.Y. (1994), Third Edition; “Current Protocols in Immunology” Volumes I-III Coligan J. E., Ed. (1994); Stites et al. (Eds.), “Basic and Clinical Immunology” (8th Edition), Appleton & Lange, Norwalk, Conn. (1994); Mishell and Shiigi (Eds.), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., Ed. (1984); “Nucleic Acid Hybridization” Hames, B. D., and Higgins S. J., Eds. (1985); “Transcription and Translation” Hames, B. D., and Higgins S. J., Eds. (1984); “Animal Cell Culture” Freshney, R. I., Ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### GENERAL MATERIALS AND EXPERIMENTAL METHODS

Mice—Female Balb/c, 8 weeks old, were obtained from Harlan Laboratories. Human MUC1 transgenic mice were kindly given by Prof. Gendler (Mayo Clinic, Arizona, USA). These mice express the human MUC1 in a pattern and level consistent with that observed in human organs including lung, mammary glands, pancreas, kidney, gall bladder, salivary glands, stomach and uterus, and is not expressed in tissues that do not normally express MUC1 such as colon, heart, liver, muscle and spleen. Moreover, these transgenic mice have been shown to be tolerant to MUC1 (13).

Epitopes used for vaccination—The first peptide [APDTRPA (SEQ ID NO:1)] inserted in the flagellin is an established humoral and cellular epitope in mouse and in human (Apostolopoulos, 1994); this recombinant vaccine is referred to as Fla-MUC1.7. The second epitope [RPAPGSTAP SEQ ID NO:2)] was selected using the bioinformatics tool SYFPEITHI database (Nussbaum A K et al. 2003; Hans-Georg Rammensee, et al., 1999)), for its higher affinity to Balb/c mice MHC class I (H2d) compared to the peptide set forth by SEQ ID NO: 1, but also present a non negligible affinity with many human MHC class 1 molecule available on the SYFPEITHI database; the second recombinant vaccine is referred as Fla-MUC1.9. Additionally, the sequence covering all the epitopes of the TR in mouse (Balb/c) and in human (MHC I alleles available) was defined using the SYFPEITHI database as: GVTSAPDTRPAPGSTAPPAHGVTSA (SEQ ID NO:5); the third recombinant vaccine is referred to as Fla-MUC1.25. However, since it was not known whether it is possible to insert in the flagellin a sequence with such a size (25 amino acids) without affecting the structure of the flagella, the following 3 constructs were designed: Fla-MUC1.20 (a flagellin including one TR; GVTSAPDTRPAPGSTAPPAH; SEQ ID NO:6), Fla-MUC1-22 (a flagellin including GVTSAPDTRPAPGSTAPPAHGV; SEQ ID NO:7, which includes one TR and 2 amino acid residues that are important in an epitope on the C terminal region of the TR) and Fla-MUC1.25 (a flagellin including GVTSAPDTRPAPGSTAPPAHGVTSA; SEQ ID NO:5).

Tumor cells—The 4T1 tumor cell culture (ATCC Accession No. CRL-2539), transfected with MUC1 form the MUC1 variant of 4T1 (4T1-MUC1) was kindly provided by Pr. Allen, University of Alberta, Edmonton, Canada). 4T1-MUC1 cells (Moase E H, et al., 2001) were grown in DMEM with 10% fetal bovine serum supplemented with non essential amino acid, thioguanine 60 μM, geneticin 400 μg/ml and antibiotic (penicillin and streptomycin) and amphotericin. MUC1 expression on 4T1-MUC1 cell culture and on large tumor, at 30 to 40 days post-implantation in Balb/c mice was determined by FACS (data not shown), and showed that those cells are stable transfectant.

Preparation of the recombinant flagellins—The following oligonucleotides were synthesized:

a—The oligonucleotide coding for 7 amino acid [APDTRPA (SEQ ID NO:1)] of the tandem repeat (TR) of MUC1 named MUC1.7, which represent the immunodominant region of MUC1: 5′-GCT CCG GAT ACC CGT CCG GCT GAT-3′ (SEQ ID NO:3). Note that the last 3 nucleic acids of SEQ ID NO:3 (GAT) were added to the MUC1.7 coding sequence (nucleic acids 1-21 of SQE ID NO:3) in order to preserve the EcoRV restriction site in the flagellin sequence.

b—The oligonucleotide coding for 9 amino acids included in the tandem repeat of MUC1 which [RPAPGSTAP (SEQ ID NO:2)] is named MUC1.9: 5′-AGA CCG GCT CCG GGT AGC ACC GCT CCG GAT-3′ (SEQ ID NO:4). Note that the last 3 nucleic acids of SEQ ID NO:4 (GAT) were added to the MUC1.9 coding sequence (nucleic acids 1-27 of SQE ID NO:3) in order to preserve the EcoRV restriction site in the flagellin sequence.

c—The oligonucleotide coding for the tandem repeat of MUC1 which (GVTSAPDTRPAPGSTAPPAH; SEQ ID NO:6) is named MUC1.20: 5′-GGCGTGACCTCGGCGCCGGATACCCGCCCGGCGCCGGGCTCGACCGCGCC GCCGGCGCAT-3′ (SEQ ID NO: 23).

d—The oligonucleotide coding for the tandem repeat of MUC1 which (GVTSAPDTRPAPGSTAPPAHGV; SEQ ID NO:7) is named MUC1.22: 5′-GGCGTGACCTCGGCGCCGGATACCCGCCCGGCGCCGGGCTCGACCGCGCC GCCGGCGCATGGCGTG-3′ (SEQ ID NO: 24).

e—The oligonucleotide coding for the tandem repeat of MUC1 which (GVTSAPDTRPAPGSTAPPAHGVTSA; SEQ ID NO:5) is named MUC1-25: 5′-GGCGTGACCTCGGCGCCGGATACCCGCCCGGCGCCGGGCTCGACCGCGCC GCCGGCGCATGGCGTGACCTCGGCG-3′ (SEQ ID NO: 25).

Codon usage was according to the sequence of the flagellin gene, with minor modification to create EcoRV restriction sites in oligonucleotides encoding MUC1.7 and MUC1.9 as described hereinabove.

The plasmid vector carrying the flagellin gene from Salmonella munchen (pLS408; plasmid obtained from the lab of Stocker from C. Jacob; Newton S M, Jacob C O, Stocker B A, 1989, Science. 244: 70-2) was used for the expression of the epitopes MUC1-7, MUC1-9, MUC1.20, MUC1.22 and MUC1.25. The insertion site for MUC1.7 and MUC1.9 in the flagellin amino acid sequence was the EcoRV restriction site (nucleotides 349-354 of SEQ ID NO:17) which is shown in bold in FIG. 15a. The insertion site for MUC1.20, MUC1.22 or MUC1.25 included the AgeI sticky ends and EcoRV blunt ends (the restriction sites are shown in bold in FIG. 15b).

Construction of the Fla-MUC1 plasmids which include a MUC1 insert of 20, 22 or 25 amino acids—The pLS408 plasmid was modified to include an AgeI restriction site (nucleotides 313-318 of SEQ ID NO:28; shown in bold in FIG. 15b). The modified plasmid was subjected to double digestion with AgeI and EcoRV.

For the construction of the Fla-MUC1.25 chimeric polypeptide, the MUC1.25 insert [including the sequence encoding MUC1.25 (SEQ ID NO:25) and an additional sequence of the flagellin coding sequence flanking between the AgeI and EcoRV restriction sites] was obtained after annealing of the MUC1.25 sense:

5′ CC GGT ACA GAT GGC GTG ACC TCG GCG CCG GAT ACC CGC CCG GCG CCG GGC TCG ACC GCG CCG CCG GCG CAT GGC GTG ACC TCG GCG 3′ (SEQ ID NO:29) [note that the original AT nucleotides were mutated to CC nucleotides (underlined) in order to create the AgeI restriction site]; and MUC1.25 antisense:
5′ CGC CGA GGT CAC GCC ATG CGC CGG CGG CGC GGT CGA GCC CGG CGC CGG GCG GGT ATC CGG CGC CGA GGT CAC GCC ATC TGT A 3′ (SEQ ID NO:30). oligonucleotides. The MUC1.25 insert was then ligated into the AgeI/EcoRV cut pLS408 plasmid.

For the construction of the Fla-MUC1.20 chimeric polypeptide, the MUC1.20 insert [including the sequence encoding MUC1-20 (SEQ ID NO:23) and an additional sequence of the flagellin coding sequence flanking between the AgeI and EcoRV restriction sites] was obtained after annealing of the MUC1.20 sense: 5′ CC GGT ACA GAT GGC GTG ACC TCG GCG CCG GAT ACC CGC CCG GCG CCG GGC TCG ACC GCG CCG CCG GCG CAT 3′ (SEQ ID NO:33) [note that the original AT nucleotides were mutated to CC nucleotides (underlined) in order to create the AgeI restriction site] and the MUC1.20 antisense: 3′ A TGT CTA CCG CAC TGG AGC CGC GGC CTA TGG GCG GGC CGC GGC CCG AGC TGG CGC GGC GGC CGC GTA 5′ (SEQ ID NO:34) oligonucleotides. The MUC1.20 insert was then ligated into the AgeI/EcoRV cut pLS408 plasmid.

For the construction of the Fla-MUC1.22 chimeric polypeptide, the MUC1.22 insert [including the sequence encoding MUC1.22 (SEQ ID NO:24) and an additional sequence of the flagellin coding sequence flanking between the AgeI and EcoRV restriction sites] was obtained after annealing of the MUC1.22 sense: 5′ CC GGT ACA GAT GGC GTG ACC TCG GCG CCG GAT ACC CGC CCG GCG CCG GGC TCG ACC GCG CCG CCG GCG CAT GGC GTG 3′ (SEQ ID NO:35) [note that the original AT nucleotides were mutated to CC nucleotides (underlined) in order to create the AgeI restriction site] and the MUC1.22 antisense: 3′ A TGT CTA CCG CAC TGG AGC CGC GGC CTA TGG GCG GGC CGC GGC CCG AGC TGG CGC GGC GGC CGC GTA CCG CAC 5′ (SEQ ID NO:36) oligonucleotides. The MUC1.22 insert was then ligated into the AgeI/EcoRV cut pLS408 plasmid.

The recombinant plasmids were transformed into E. coli JM101 competent cells by heat chock. Plasmids from positive colonies were purified and used to transform a flagellin negative live vaccine strain (an Aro A mutant) of S. Dublin SL5928 by electroporation. The transformed S. Dublin cells were selected for Ampicillin resistance, motility under the light microscope and growth in semisolid agar plates. The flagella comprising the hybrid flagellins were detached from the bacteria using acidic cleavage as described elsewhere (Ibrahim G. F. et al., 1991). Briefly, bacterial host cells expressing the recombinant flagellin-MUC1 polypeptide were collected from agar plates containing selective agents (Ampicillin) and grown overnight in Luria broth (LB) in the presence of the same selective agents. Cells were then collected by centrifugation (15 minutes at 6000 rpm) and pellets were resuspended in PBS. Acid cleavage of the flagellas was performed by reducing the pH (to pH=2) using Chloridric acid (6 to 10 N) under stirring during 30 minutes. Following acid cleavage, the supernatant from two successive centrifugations (the first one for 15 minutes at 9000 rpm and the second one at 19500 rpm for one hour) was subjected to ammonium sulfate precipitation (35%) and stirring over night at 4° C., following which the ammonium sulfate—containing supernatant was centrifuged 10 minutes at 10,000 rpm at 4° C., resulting in a pellet containing the recombinant flagella. The recombinant flagella pellet was resuspended in PBS and dialyzed against PBS in the presence of activated charcoal. The purity of the isolated peptides was assessed by SDS-PAGE.

The recombinant flagellin harboring MUC1-7 was denoted Fla-MUC1.7 (the amino acid sequence of the chimeric polypeptide comprises SEQ ID NO:8), the flagellin carrying MUC1.9 was denoted Fla-MUC1.9 (the amino acid sequence of the chimeric polypeptide comprises SEQ ID NO:9), the flagellin carrying MUC1.20 was denoted Fla-MUC1.20 (the amino acid sequence of the chimeric polypeptide comprises SEQ ID NO:26), the flagellin carrying MUC1.22 was denoted Fla-MUC1.22 (the amino acid sequence of the chimeric polypeptide comprises SEQ ID NO:27) and the flagellin carrying MUC1.25 was denoted Fla-MUC1.25 (the amino acid sequence of the chimeric polypeptide comprises SEQ ID NO:22). The flagellin bearing an epitope of the nucleoprotein of influenza NP 147-158 (TYQRTRALVRTG; SEQ ID NO:21) was denoted Fla-NP and serves as a control vaccine (the amino acid sequence of the chimeric polypeptide comprises SEQ ID NO:20).

Immunization procedures and tumor challenge—In therapeutic experiments, mice were implanted subcutaneous (s.c.) on the back with 1.5 million 4T1-MUC1 cells in a total volume of 100 μl in PBS. Once palpable tumors were detected and measurable at 7 to 14 days post-implantation, 20 (for the dose experiment depicted in FIG. 7), 50 or 100 μg (depending on the experiment) of the recombinant flagellins in PBS were administrated to the mice bearing the tumor. Tumor growth was monitored twice a week using calliper and determining tumor volume using the equation: volume=0.4 ab², where “a” is the larger diameter and “b” is the smaller diameter.

In prophylactic experiments, 100 μg of the recombinant flagellins with or without adjuvant (depending on the experiment) were administrated once or more (e.g., a few times) to the mice prior to tumor challenge. One month or 4 months after the last immunization, tumor challenge was performed by implanting s.c. 1.5×10⁶ of 4T1-MUC1 cells. Tumor growth was monitored as mentioned above.

For the lymphocyte proliferation assay, mice were immunized 3 or 4 times with 100 μg of the recombinant flagellins; first immunization was in complete Freund's adjuvant and immunization boosts in incomplete Freund's adjuvant.

Anti-MUC1 antibody detection—BSA coupled to the 20 amino acid peptide of the tandem repeat of MUC1 (GVTSAPDTRPAPGSTAPPAH; SEQ ID NO:6) or the residues peptide as such (i.e., not coupled to BSA) were absorbed overnight at 4° C. to ELISA plates in carbonate buffer (pH 9.6). Plates were washed twice in PBS Tween (0.1%). Blocking was performed for one hour at 37° C. with PBS containing 1% bovine serum albumin (BSA). Serial dilution of sera of mice, as specified, were added (50 μl/well) and incubated for 2 hours at 37° C. After four washes in PBS-Tween, goat anti-mouse IgG antibodies, conjugated to horseradish peroxidase (HRP) or goat anti-mouse IgG3 (also conjugated to HRP) was used as a second antibody and incubated for 2 hours at 37° C. Finally, 3, 3′, 5, 5′ tetramethylbenzidine (TMB) was added as substrate, and 1N HCl was used to stop the reaction. The plate was read at 450 nm.

Lymphocytes proliferation assay—The ability of cells to proliferate in vitro in response, to incubation with various antigens was monitored as followed. Lymphocyte from spleen (splenocytes) and lymph nodes (lymphocytes) removed aseptically several days (depending on the schedule) after the last immunization, and single cell suspension was prepared in RPMI1640 medium containing 2 mM L-glutamine, antibiotics, 5×10⁻⁵ M 2-mercaptoethanol and 5% fetal calf serum (FCS). The cells were cultured in 0.2 ml in the presence of the antigens, and the proliferation response was evaluated 3, 5 and 7 days later by pulsing the cells for 18 hours with [³H]thymidine and monitoring the incorporated radioactivity. Each test was performed in six plicate.

Supernatant for the culture were taken at different days to detect the presence of different cytokines using R&D kit (ELISA sandwich).

Metastasis assay—This assay was performed in three steps:

A—Preparation of Organs:

Lungs were removed, swirled using forceps in order to extract any remaining blood. Then, lungs were cut into small pieces using curved scissors and transferred into a 15 ml conical tube containing 2.5 ml (=5 mg) of collagenase type IV and 30 units elastase and incubated for 75 minutes on platform rocker at 4° C.

Liver was removed, swirled using forceps in order to extract any remaining blood. Then, lungs were cut into small pieces using curved scissors which were then transferred into a 15 ml conical tube containing 2.5 ml (5 mg) collagenase type 1 and 2.5 ml (5 mg) hyaluronidase. The samples were placed at 37° C. for 20 to 30 minutes on a platform rocker.

B—Wash Enzyme Digested Sample:

The volume of the samples was brought to 10 ml with 1×HBBS (Hank's balanced salt solution). Large chunks of undigested tissue were removed by filtration with 70 μm nylon cell strainer, samples were centrifuged for 5 minutes at 1500 rpm at room temperature and pellet were washed 3 times with 1×HBBS. The pellets were then resuspended in 10 ml medium (same medium described above for 4T1-MUC1 cells) and samples were pelletted (either neat or diluted) and incubated for 10-14 days in a 37° C. tissue culture incubator in the presence of 5% CO₂ (avoiding any disturbance to the plate).

C—Harvesting Clonogenic Metastatic Colonies:

Culture medium was discarded from tissue culture plates. Cells were fixed by adding 5 ml methanol at room temperature for 5 minutes under a chemical hood. Colonies should turn white. Methanol was discarded with 5 ml distilled water. Then 5 ml 0.03% methylene blue was added at room temperature for 5 minutes and finally plates were washed with 5 ml distilled water and colonies were counted.

### Example 1

### Prophylactic Vaccination with Fla-MUC1.7 Inhibits Tumor Growth

To test the effect of the Fla-MUC1.7 vaccine in preventing tumor growth, the following experimental approach was employed.

Twenty five Balb/c mice were immunized 3 times in 4 weeks intervals as followed:

Group 1 (n=9): 100 μg Fla-MUC1.7 (which comprises a chimeric polypeptide comprising SEQ ID NO:8)+complete Freund's adjuvant (CFA) (2 boost in incomplete Freund's adjuvant (IFA)

Group 2 (n=6): 100 μg Fla (as set forth by SEQ ID NO:10)+CFA (2 boosts in IFA)

Group 3 (n=7): PBS+CFA (2 boosts in IFA) Four months after the last boost, mice were implanted with 1.5×10⁶ 4T1-MUC1 cells and tumor growth was monitored.

Experimental Results

Prophylactic vaccination with Fla-MUC1.7 results in a protective effect of on tumor growth—As is shown in FIG. 1, until the 24^{th} day post-implantation, the average tumor size of the group immunized with Fla-MUC1.7 was 7 to 8 times smaller than the mice immunized only with adjuvant (p<0.01). From the 28^{th} day this ratio was still significant with a 5 to 6 times smaller tumor size in mice immunized with the Fla-MUC1.7 as compared with mice immunized only with adjuvant.

Thus, these results demonstrate the efficacy of a prophylactic vaccination with the Fla-MUC1.7 immunogene in preventing the growth of tumors. Thus, these results suggest the use of the Fla-MUC1.7 immunogene in preventing cancer especially in individuals who are predisposed to tumor formation. For example, about 10% of the breast cancer patients are due to inherited mutations in the BRCA1 or BRCA2 genes. In this high risk group of subjects, young females identified with suspected genetic predisposition (familial history of cancers, or identified mutation in BRCA1 or BRCA2 genes) are even offered prophylactic mastectomy. For this target population, the availability of a prophylactic vaccination against breast cancer would be a plausible option.

### Example 2

### Therapeutic Vaccination with Fla-MUC1 with CFA Adjuvant

While a prophylactic vaccination as described in Example 1, hereinabove, may be offered to at-risk subjects, the concept of a therapeutic vaccine is much more practical and valuable for the entire population. With this in mind, and in order to explore the effect of the chimeric Fla-MUC1.7 polypeptide on tumor growth, the following experiment was performed.

25 Balb/c female mice were s.c. implanted with 1.5×10⁶ 4T1-MUC1 cells. About 10 days post-implantation, mice were separated in two group denoted A and B: mice bearing a measurable tumor (A) and mice with palpable but not measurable tumor (B). Those two groups were used to form 4 groups of mice with the same proportion of A and B, which were immunized subcutaneously as followed:

Group 1 (8 mice): 100 μg of Fla in CFA

Group 2 (8 mice): 100 μg of Fla-MUC1.7 in CFA

Group 3 (5 mice): CFA (control group)

Group 4 (4 mice): PBS (control group)

Experimental Results

The chimeric Fla-MUC1.7 is capable of inhibiting the growth of a solid tumor—FIG. 2 depicts the tumor growth in each group at the first day of immunization (day 0) which is also the first day of measuring tumor size and at day 19 post-immunization (day 19) which is the last day of measuring tumor size before animals were euthanized according to the regulation of the IACUC due to the appearance of one tumor diameter which exceeds 1.5 cm.

As is shown in FIG. 2, at day 19, the average size of tumors in the group immunized with Fla-MUC1.7 is 4.5 times smaller than the average size of tumor in the group of mice immunized with CFA (P<0.05), and 5 times smaller than the size of tumor in the group immunized with PBS (P<0.01). Although the average tumor growth of the group immunized with Fla alone is smaller than the averages of the groups immunized with CFA and PBS, this difference is not statistically different (P>0.05).

Consequently, these results demonstrate that the chimeric Fla-MUC1.7 is capable of inhibiting the growth of a solid tumor.

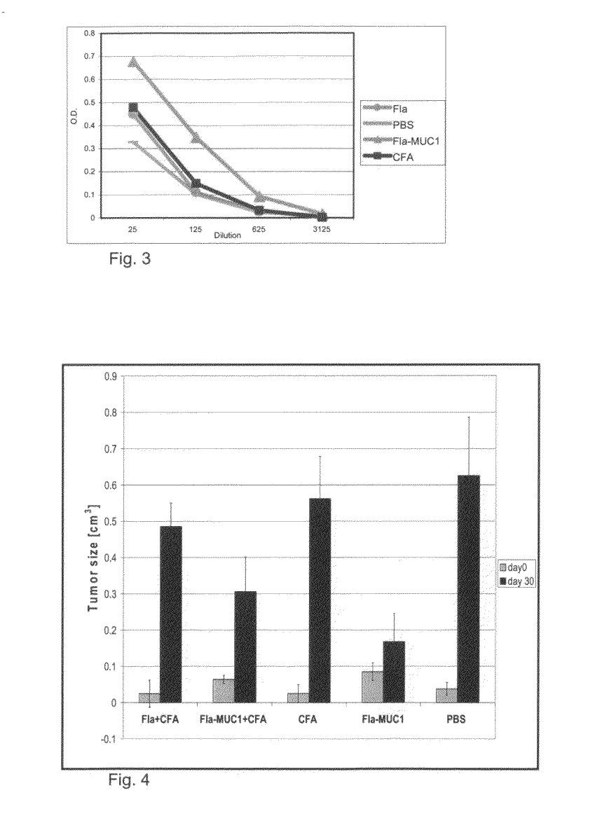

Fla-MUC1.7 immunization results in increased IgG response—The IgG anti-MUC1 response was also investigated in the treated mice. As is shown in FIG. 3, at a dilution of 1/125, the IgG anti-MUC1 titer is 3.2 fold higher in group 2 (immunized with Fla-MUC1.7) than the IgG anti-MUC1 in group 1 (immunized with Fla-CFA) or 3 (immunized with CFA).

Thus, in view of these results, it seems that the relative small average tumor size in the group immunized with the recombinant vaccine Fla-MUC1.7 could be at least partially due to a humoral response against the tumor.

### Example 3

### Immunization of Mice Bearing Tumor with the Recombinant Flagellin Fla-MUC1.7 is More Efficient without Adjuvant

To investigate if CFA is required for the protective effect of Fla-MUC1.7 on the tumor growth, the following experimental approach was employed.

Balb/c female mice were implanted subcutaneously with 1.5×10⁶ 4T1-MUC1 cells and one week post-implantation, the mice were immunized as followed:

Group 1 (8 mice): 100 μg of Fla in CFA

Group 2 (8 mice): 100 μg of Fla-MUC1.7 in CFA

Group 3 (8 mice): 100 μg of Fla

Group 4 (8 mice): 100 μg of Fla-MUC1.7

Group 5 (4 mice): CFA (control group)

Group 6 (4 mice): PBS (control group)

### Experimental Results

Immunization of mice bearing tumor with the recombinant flagellin Fla-MUC1.7 is more efficient in slowing down tumor growth in the absence of adjuvant—FIG. 4 depicts the growth of tumor in each group at the first day of immunization (day 0) and 30 days post-immunization (day 30). Because 6 of the 8 mice of the group 3 died 2 days post-immunization probably due to contamination with endotoxins in the preparation, this group is not represented in the graph.

At day 30, the mice immunized with Fla-MUC1-7 with CFA show significant (P<0.05) 2 times smaller average tumor size than the control groups, which confirm the results obtained in the first experiment described in Example 2 hereinabove.

Interestingly, the group immunized with Fla-MUC1.7 (without CFA), displayed an average tumor size which is 3 times smaller than in the control group with higher significance (P<0.01).

Altogether, these results demonstrate that not only CFA is not required for the protective effect of Fla-MUC1-7 on tumor growth, which is supported by the already described adjuvant effect of the flagellin (Levi et al., 1996; Cuadros C. et al. 2004), but rather immunization of mice bearing tumor with the recombinant flagellin Fla-MUC1.7 seems to be more efficient in slowing down tumor growth than the chimeric molecule with adjuvant (CFA). Thus, the further experiments were carried out in the absence of adjuvant.

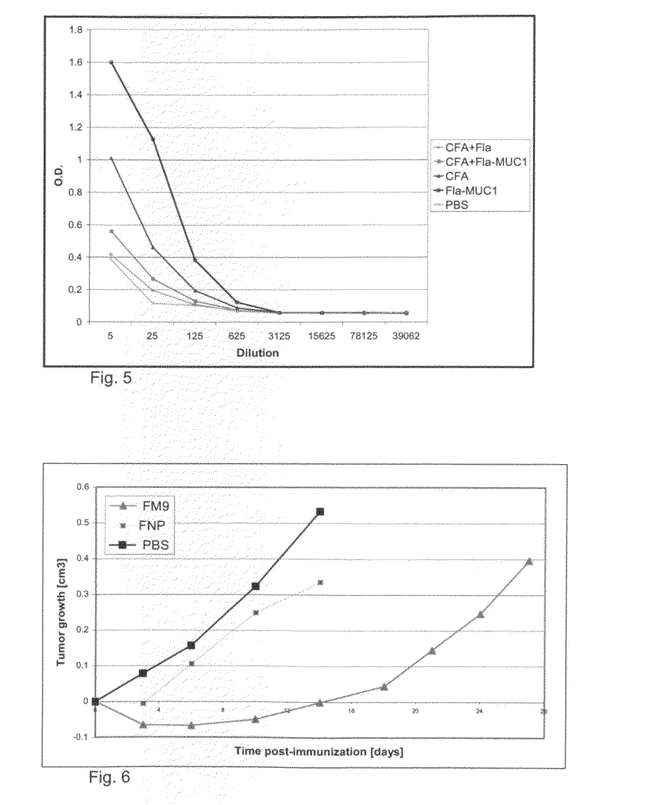

The IgG response anti-MUC1 is higher in mice immunized with Fla-MUC1.7 than in mice immunized with Fla-MUC1.7 with CFA or with CFA alone—IgG response anti-MUC1 was assessed in one mouse per group. Each of the mice used for measuring the IgG response was selected in order that its tumor size would be as more representative as possible as the average tumor size of its group. As is shown in FIG. 5, the mouse immunized with Fla-MUC1-7 presents a very high titer of IgG anti-MUC1 comparing to the mice from control groups or even comparing to the mouse immunized with Fla-MUC1.7+CFA. On the other hand the mouse immunized with Fla-MUC1.7+CFA does not display higher antibody titer than the mouse from the control groups as observed in the previous experiment. Moreover, the mouse immunized with CFA shows a high antibody titer, as its tumor size is not different than other control groups.

Consequently, a cellular immune response against the tumor might be induced by Fla-MUC1.7 (+/−CFA), and might be responsible for the protective effect of this chimeric molecule on tumor growth. Thus, lymphocytes proliferation assay was performed on splenocytes from the same mice selected for the anti-MUC1 humoral response. However, no proliferation in the lymphocyte proliferation assay was observed towards a MUC1 peptide (GVTSAPDTRPAPGSTAPPAH; SEQ ID NO:6, 20 amino acid from the tandem repeat of MUC1) after 3 days of culture and none of the cytokines tested (IL-4, IL-5, IL-10, IL-2, IFNγ, TNFα) were detected in the supernatant taken after 24 and 48 hours of culture. Further evaluation of cytokines involves (specially Th1 induced) in the therapeutic effect induced by the present invention will be carry out using more sensitive techniques such as ELISPOT or FACS intracellular staining.

Altogether, these results suggest mainly the involvement of a cellular response against the tumor induced by Fla-MUC1.7.

### Example 4

### Effect of Fla-MUC1.9 and Immunization Dosage on Tumor Growth

Experimental Results

Mice bearing tumors and vaccinated with the Fla-MUC1.9 exhibited a significantly reduced tumor growth and could be kept alive twice longer than the control group—To examine the effect of the recombinant chimeric Fla-MUC1.9 on tumor growth, the following experiment was performed.

32 Balb/c female mice (8 weeks old) were implanted subcutaneously with 1.5×10⁶ 4T1-MUC1 cells. Ten days post-implantation, mice were immunized subcutaneously as followed:

Group 1 (15 mice): 100 μg of Fla-MUC1.9 (which comprises a chimeric polypeptide comprising SEQ ID NO:9)

Group 2 (8 mice): Fla-NP (control group)

Group 4 (9 mice): PBS (control group) Tumor growth was monitored until mice were euthanized according to the regulation of the Weizmann Institute.

Fourteen days post-immunization mice from the two control groups had to be euthanized, whereas the average tumor growth of the mice immunized with Fla-MUC1.9 were just reaching back the initial size of the tumor as of the day of immunization (FIG. 6). Thus, on day 14, mice immunized with Fla-MUC1.9 presented a tumor size more than 5 times smaller than the tumor size of the PBS control group (p<0.01). Moreover, the mice vaccinated with Fla-MUC1.9 were euthanized at day 27 according to the same criteria used to sacrifice the mice from the control groups on day 14. Consequently, this experiment shows that mice bearing tumor which were immunized with Fla-MUC1.9 could be kept alive twice longer than the one of the control group.

Immunization dose has a significant therapeutic effect—The effect of different doses of Fla-MUC1.7 and Fla-MUC1.9 on tumor growth was explored. For this purpose, 48 female Balb/c mice (8 weeks old) were implanted subcutaneously with 1.5×10⁶ 4T1-MUC1 cells. Ten days post-implantation, mice were immunized as follows:

Group 1 (6 mice): 20 μg of Fla-MUC1.7

Group 2 (6 mice): 20 μg of Fla-MUC1.9

Group 3 (6 mice): 50 μg of Fla-MUC1.7

Group 4 (6 mice): 50 μg of Fla-MUC1.9

Group 5 (6 mice): 100 μg of Fla-MUC1.7

Group 6 (6 mice): 100 μg of Fla-MUC1.9

Group 7 (12 mice): PBS (control)

Tumor growth was monitored until mice were euthanized according to the regulation of the Weizmann Institute. As is shown in FIG. 7, while immunization of mice with 50 or 100 μg/per mouse of either Fla-MUC1.7 or Fla-MUC1.9 resulted in a significant inhibition of tumor growth as compared to PBS control, immunization of mice with 20 μg/per mouse of Fla-MUC1.7 per mouse was not sufficient in order to slow down tumor growth. On the other hand, immunization of mice with 20 μg of Fla-MUC1.9 resulted in efficient inhibition of tumor growth.

Thus, these results demonstrate that immunization with Fla-MUC1.9 is more efficient than with the Fla-MUC1.7. In addition, the results demonstrate that the dose used for immunization has a significant therapeutic outcome.

### Example 5

### Effects of Co-Administration of Fla-MUC1.7 with Fla-MUC1.9 and Multiple Immunizations on Tumor Growth

Experimental Designs

Testing the efficacy of co-administration of vaccines on tumor growth—In order to evaluate the combined effect of the two MUC1 vaccines on the growth of tumor, the following experiment was performed.

45 female Balb/c mice (8 weeks old) were implanted subcutaneously with 1.5×10⁶ 4T1-MUC1 cells. Ten days post-implantation, mice were immunized as follows:

Group 1 (10 mice): 50 μg of Fla-MUC1.7

Group 2 (10 mice): 50 μg of Fla-MUC1.9

Group 3 (10 mice): 50 μg of Fla-MUC1.7+50 μg of Fla-MUC1.9

Group 4 (5 mice): 50 μg of Fla-NP

Group 5 (10 mice): PBS (control)

Tumor growth was monitored until mice were euthanized according to the regulation of the Weizmann Institute.

Testing the efficacy of multiple immunizations with a vaccine on tumor growth—The following experiment was performed in order to test the efficacy of multiple immunizations on tumor growth:

45 female Balb/c mice (8 weeks old) were implanted subcutaneously with 1.5×10⁶ 4T1-MUC1 cells. The first immunization was done, as in the previous experiments, ten days post-implantation, as followed:

Group 1 (10 mice): 50 μg of Fla-MUC1.7

Group 2 (10 mice): 50 μg of Fla-MUC1.9

Group 3 (10 mice): 50 μg of Fla-MUC1.7+50 μg of Fla-MUC1.9

Group 5 (10 mice): PBS (control)

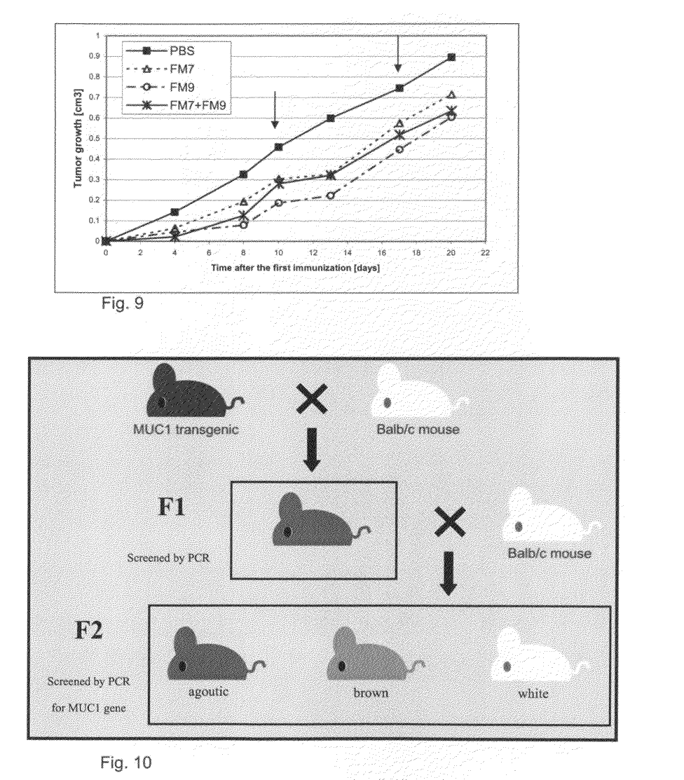

The same immunization was repeated 10 days (second immunization) and 17 days (third immunization) after the first immunization (notified by arrows on the FIG. 9).

Tumor growth was monitored until mice were euthanized according to the regulation of the Weizmann Institute.

Experimental Results

Co-administration of Fla-MUC1.7 and Fla-MUC1.9 as compared to administration of each vaccine alone—As is shown in FIG. 8, the tumor growth in mice immunized with Fla-MUC1.7+Fla-MUC1.9 (FM7+FM9) is significantly different than the group of mice immunized only with one of the two vaccines (FM7 or FM9) on the last day of the experiment.

Moderate effect of multiple immunizations with the recombinant flagellins on tumor growth—The results described above were obtained while immunizing only once the mice bearing the 4T1-MUC1 tumor. Consequently, it became obvious to explore the possibility that several immunizations would benefit to the treatment. As is shown in FIG. 9, following the second immunization at day 10, tumor growth is slowed down (p<0.05), whereas the third immunization didn't exert any effect. However, in a repeated experiment the effect of the second immunization was limited. These results may suggest that MUC1, as a glycoprotein, may be recognized as a foreign antigen by the immune system of the Balb/c mice. In addition, other studies showed that human MUC1 is highly immunogenic in mice (Taylor-Papadimitriou et al. 2001).

### Example 6

### The Fla-MUC1.7 Vaccine Inhibits Tumor Growth and Metastasis in MUC1 Transgenic Mice

The results presented above were performed in Balb/c mice bearing a tumor expressing a human protein which is highly immunogenic in mice (Taylor-Papadimitriou et al. 2001). Consequently, an important question remains: do Fla-MUC1.7 and Fla-MUC1.9 boost the immune response induced by MUC1 on the surface of the tumor cells, and consequently depend on it, or do those vaccines induce an immune response by themselves?

To address, this question the present inventors have tested the therapeutic effect of the two recombinant vaccines on tumor growth in human MUC1 transgenic mice (kindly given by Professor S. Gendler, Mayo clinic, Rochester). These mice have been shown to express the human MUC1 in the pattern and in the level consistent to that observed in human, and thus to be tolerant to MUC1 (Rowse G J, et al., 1998; Nussbaum A K, et al., 2003). This model provides a valuable pre-clinical model involving MUC1. Moreover, the cell line 4T1-MUC1 is expected to induce spontaneous metastasis to the lung and to the liver in the MUC1 transgenic mice, as the parental cell line 4T1 does in Balb/c, offering in this way the possibility to assess the therapeutic activity of Fla-MUC1.7 and Fla-MUC1.9 on metastatic disease.

Experimental Design

Crossing between Babl/c and MUC1 transgenic C57/Black6 mice—Since the MUC1 transgenic mice are of C57/Black6 genetic background, and since 4T1-MUC1 cell line is growing in Babl/c, the MUC1 transgenic mice were crossed with Babl/c mice, in order to obtain generations of mice (F1 and F2 minimum) that could potentially accept the cell line of interest. Thus, before testing the effect of Fla-MUC1.7 on tumor growth in MUC1 transgenic mice, the capacity of 4T1-MUC1 cell line to grow in the MUC1 transgenic mice was assessed.

Establishment of the model—Four F1 mice, positive for MUC1, were implanted subcutaneously with 10⁵ (group 1) and 5×10⁵ (group 2) 4T1-MUC1 cell line (2 mice per group). 8 days post-implantation only 1 mouse from group 2 presented a tumor, and the second mice presented a tumor one week afterwards. 26 days post implantation mice were euthanized and lung and liver micrometastasis were examined. Results are displayed in Table 1, hereinbelow.

TABLE 1
Table 1: Evaluation of tumor size and liver micrometastasis in
F1 MUC1 transgenic mice implanted with 4T1-MUC1 cell line.

| | Mouse 1 group 2 | Mouse 2 group 2 |
|---|---|---|
| Tumor size (cm³) | 1.4 | 0.8 |
| Liver micrometastasis | 0 | 0 |

Since 4T1-MUC1 cell line is growing in MUC1 transgenic mice and is inducing metastasis to the lung, the effect of Fla-MUC1-7 on tumor growth in transgenic mice could be tested. However, more cells are to be implanted in order to try avoiding the difference of growing of the tumor.

Immunization protocol of Fla-MUC1.7 in human MUC1 transgenic mice following implantation of 4T1-MUC1 cells—The experiment was carried out with the F2 generation (since more mice were available) obtained by successive crossings as illustrated in FIG. 10. Mice used for crossing were selected by PCR for the presence of MUC1 gene (data not shown).

Twelve F2 mice (12 months old) were implanted subcutaneously with 1.5×10⁶ 4T1-MUC1 cells. 10 days post-implantation, mice were immunized as followed:

Group 1 (6 mice): 100 μg of Fla-MUC1.7

Group 2 (6 mice): 100 μg Fla-NP (control group)

Due to differences of tumor size between the 2 groups (group 2 presented higher tumor average comparing to group 1) at day 0 (day of immunization), normalization was applied. The ratio between the tumor size at different time points and the tumor size at day 0 was calculated and is shown in FIG. 11.

Experimental Results

Immunization with the Fla-MUC1.7 vaccine has a significant therapeutic effect on tumor growth—As is shown in FIG. 11, at day 16, the group of mice immunized with Fla-MUC1.7 presents an average tumor size which is significantly smaller (by 4 times, p<0.01) than the group immunized with the flagellin carrying a non relevant epitope (Fla-NP).

At the same date (day 16), one mouse per group, representative of the average tumor size of its respective group, was sacrificed subjected to evaluation of metastasis number and clonogenity.

Treatment of MUC1 transgenic mice with the Fla-MUC1.7 vaccine resulted in a significant decrease in lung and liver metastasis following implantation of 4T1-MUC1 cells—Further evaluation of F2 MUC1 transgenic mice (FIG. 10) which were implanted with the 4T1-MUC1 cell line revealed the presence of both lung and liver metastases (Table 2, hereinbelow). In addition, treatment of the implanted MUC1 transgenic mice with the Fla-MUC1.7 vaccine revealed a significant decrease in the number of both lung and liver metastases in the Fla-MUC1-7—treated animals (Table 2, hereinbelow).

TABLE 2
Table 2: Assessment of the number of lung and liver metastasis
in F2 MUC1 transgenic mice implanted with 4T1-MUC1 cell line.

| | Lung metastasis | Liver metastasis |
|---|---|---|
| Mouse 1 (Fla-MUC1.7) | 1 | 7 |
| Mouse 2 (Fla-NP) | 20 | 11 |

The mouse immunized with Fla-MUC1.7 presents only one metastasis in the lung, and 7 metastases in the liver; as the mouse immunized with Fla-NP presents 20 metastases in the lung and 11 metastases in the liver. Thus, it seems that Fla-MUC1-7 induces an immune response protecting the mice from the tumor growth and subsequently from the appearance of lung and liver metastasis. However, the size of the metastasis counted in these 2 mice in each organ was different, indicating that the metastasis appeared at different times.

In order to accurately evaluate the number of metastasis within each organ, a clonogenic metastasis assay on liver and lung from the treated animals was performed. For this assay the organ of interest (e.g., lung, liver) was digested with a set of enzymes (Collagenase type IV and elastase for lung and collagenase type I and hyaluronidase for liver, for further details see “General Materials and Experimental Methods” hereinabove), in order to obtain a single cell suspension that was further cultured in a thioguanine-containing medium in which only the 4T1-MUC1 cells can grow. The results of this assay for the lung tissue are displayed in Table 3, hereinbelow.

TABLE 3
Table 3: Number of clonogenic metastasis in the lung

| | Number of clonogenic metastasis in the lung |
|---|---|
| Mouse 1 (Fla-MUC1.7) | 538 |
| Mouse 2 (Fla-NP) | 5344 |

Thus, it is clear from the clonogenic metastasis assay that the mouse immunized with Fla-MUC1.7 exhibits 10 times less clonogenic metastasis than the mouse immunized with Fla-NP (control).

### Example 7

### Immune Response in MUC1 Transgenic Mice or in Balb/c Mice Treated with the Fla-MUC1 Vaccine

Experimental Results

### (1) Evaluation of the Humoral Immune Response Induced by the Recombinant Flagellin Molecules in Mice Bearing Tumors

Immunization of MUC1 transgenic mice bearing tumors with Fla-MUC1.7 does not induce higher IgG response to MUC1 than immunization with Fla-NP—Serum was isolated from blood and IgG anti-MUC1 titer were assessed for the 2 transgenic (Tg) mice and two mice (Balb/c) from the first experiment described in Example 3, hereinabove, were used as control and as indicator (FIG. 12).

Surprisingly, not only the transgenic mouse immunized with Fla-MUC1.7 do not exhibit a higher antibody titer than the transgenic mouse immunized with Fla-NP which could have been the reason for protecting the mice from tumor growth (and from metastasis appearance), like in the experiment described in Examples 2 and 3 (FIGS. 3 and 5) for Balb/c mice, but this transgenic mice show much less antibody than the control mice. Thus, the humoral response doesn't seem to explain the protective effect of Fla-MUC1-7, and probably a cellular immune response is involved.

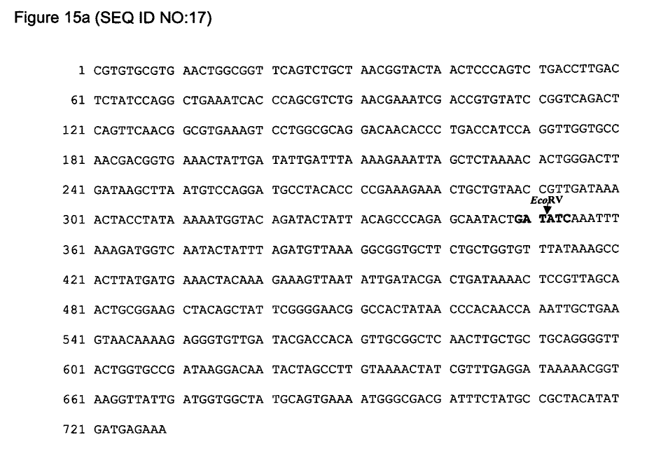

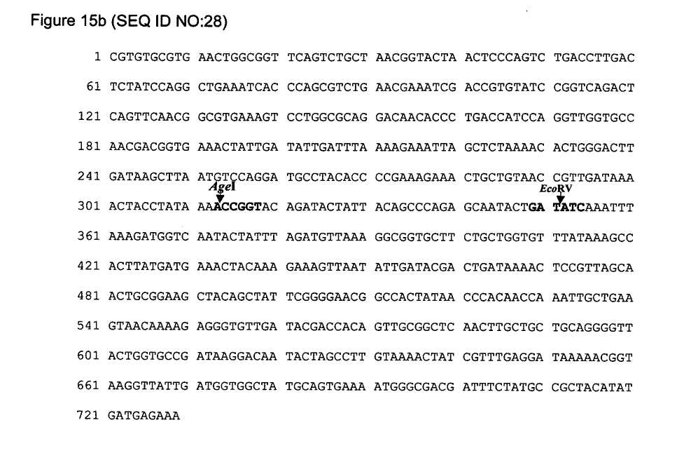

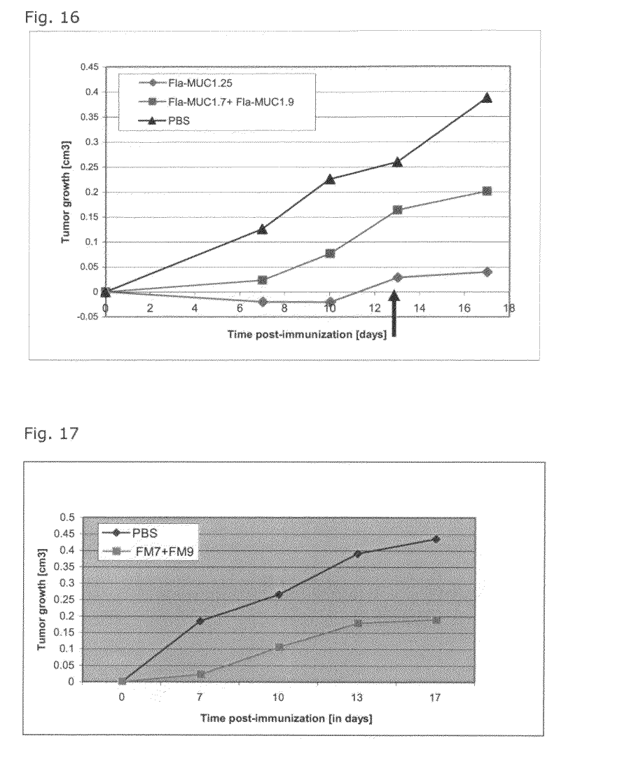

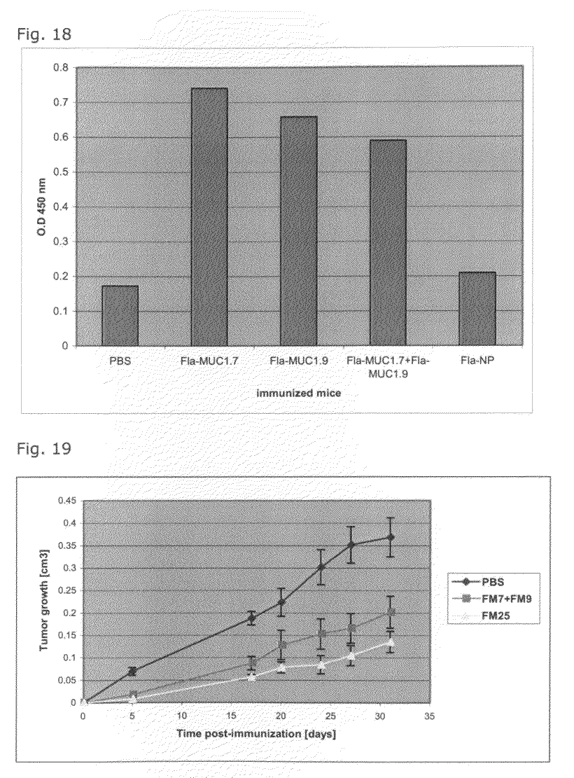

Evaluation of the antibody isotype induced by the recombinant flagellin molecules in Balb/c mice baring tumors—Serum from Balb/c mice bearing tumors and immunized twice within 10 days intervals with Fla-MUC1.7 or Fla-MUC1.9 or Fla-MUC1.7+Fla-MUC1.9 recombinant flagellas display a higher IgG 3 antibody titer than mice immunized with Fla-NP or PBS (FIG. 18). Such antibody isotype are produced in response to INFγ induced in Th1 response.

### (2) Evaluation of the Humoral Immune Response Induced by the Recombinant Flagellin Molecules in Mice not Bearing Tumors

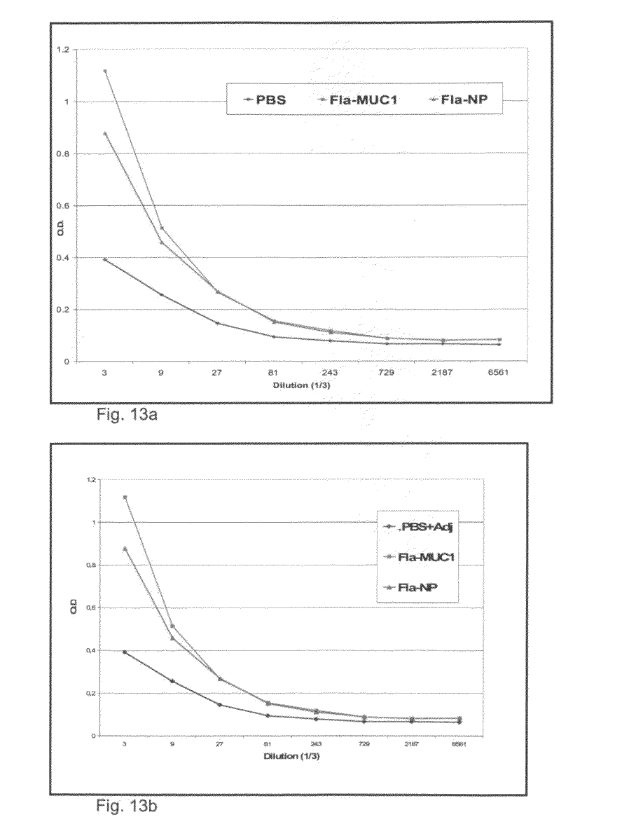

To evaluate the effect of the different recombinant flagellins on the humoral response independently of the presence of tumor, IgG titers anti-MUC1 were assessed in mice not bearing tumors which immunized 3 (FIG. 13a) and 4 (FIG. 13b) times, in 4 weeks intervals, with 100 μg of different recombinant flagellins with adjuvant (first immunization in CFA and other boosts in IFA). Mice were sacrificed 8 to 10 days after the last immunization, serum, spleen and lymph nodes were removed for IgG titrating and lymphocyte proliferation assay (see below).

Unexpectedly, the antibody titer was quite similar in all mice immunized with the different recombinant flagellins.

(3) Evaluation of the cellular immune response in mice not bearing tumors induced by the recombinant flagellin molecules—More than 10 different lymphocyte proliferation assay protocols (e.g., immunization schedule, time of culture, antigen concentration in vitro), were performed in Balb/c mice without finding any proliferation in vitro which is stimulated by MUC1 peptide (20 amino acids of the tandem repeat of MUC1; SEQ ID NO:6) after immunization with Fla-MUC1.7 and neither IFNγ nor TNFα were detected in the supernatant taken after 24 and 48 hours of culture (data not shown).

Briefly, female Babl/c mice (8 weeks old) were immunized following different schedule (from one to 3 times of 50 to 100 μg of recombinant flagellin per mouse with or without adjuvant). After a variable period of time (from 10 days to 3 weeks) splenocytes (from the spleen) and lymphocytes (from lymph nodes inguinales, brachials and axillaries) were cultured with MUC1 peptide (5 μg/well [Taylor-Papadimitriou et al. 2002]; SEQ ID NO:6), and flagellin (1 μg/well) as positive control. Proliferation rate were evaluated after several days of culture (from 3 to 7 days) by [H³] thymidine incorporation. Neither IFNγ nor TNFα were detected in the supernatant after 24 and 48 hours of culture.

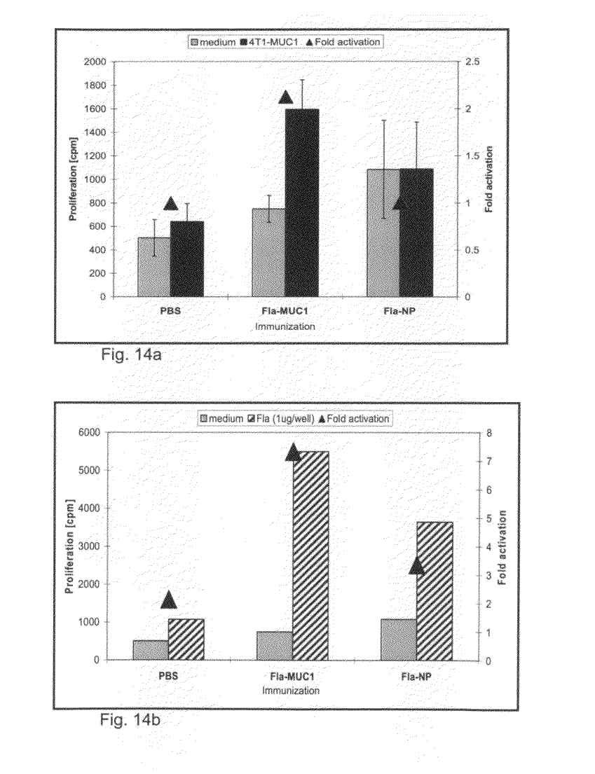

Finally, a lymphocyte proliferation assay performed on splenocytes of mice not bearing tumors 8 days after the 4^{th} immunization of 100 μg of Fla-MUC1.7 with adjuvant (first immunization with CFA and 3 boosts in IFA, 2 weeks intervals) shows a slight splenocytes proliferation to 4T1-MUC1 tumor cells after 6 days of culture (FIG. 14a). FIG. 14b displays splenocytes proliferation to the flagellin in vitro (control). Supernatant were removed after 6 days of culture in order to be further tested for the presence of different cytokines.

Altogether, these results support the involvement of a cellular response triggered by the composition of the present invention.

### Example 8

### Effects of Co-Administration of Fla-MUC1.7 with Fla-MUC1.9 on Tumor Growth in Human MUC1 Transgenic Mice

Experimental Design

Immunization protocol of Fla-MUC1.7+Fla-MUC1.9 in human MUC1 transgenic mice following implantation of 4T1-MUC1 cells—The experiment was carried out with the F1 generation obtained by crossing human MUC1 transgenic mice of C57/Black background with Balb/c mice; and mice for this experiment were selected by PCR for the presence of MUC1 gene (data not shown).

Seven F1 female mice (8-10 weeks old) were implanted subcutaneously with 1.5×10⁶ 4T1-MUC1 cells. 10 and 13 days post-implantation, mice were immunized as followed:

Group 1 (3 mice): PBS (control group)

Group 2 (4 mice): 50 μg of Fla-MUC1-7+50 μg of Fla-MUC1.9

Tumor growth was monitored by deducting the tumor size on the first day of immunization to the tumor size on each day. Results are shown in FIG. 17.

Experimental Results

Immunization with the Fla-MUC1-7+Fla-MUC1.9 vaccine results in a significant therapeutic effect on tumor growth—As is shown in FIG. 17, at day 17 post immunization, the group of mice immunized with Fla-MUC1.7+Fla-MUC1.9 presents an average tumor growth which is significantly smaller (by more than 2 times, p<0.01) than the group immunized with the PBS.

To further detect the effect of the combined vaccine (Fla-MUC1.7+Fla-MUC1.9) on the presence and/or growth of metastasis, the metastasis assay is preformed. To detect any symptoms of autoimmunity in organs expressing MUC1 (such as mammary glands, pancreas, colon etc.) immunohistochemistry, using anti Muc 1 antibodies, is performed.

Altogether, these results clearly demonstrate that the combined vaccine of Fla-MUC1.7+Fla-MUC1.9 exhibits a significant therapeutic effect in inhibiting tumor growth in MUC1 transgenic mice which were implanted with the 4T1-MUC1 cancerous cells.

### Example 9

### Development of Anti-Tumor Therapeutic Vaccines Including Additional Epitopes with MUC1 Specificity

The present inventors have designed new recombinant flagellas which cover the two epitopes MUC1.7 and MUC1.9 described above, and which include other epitopes of the tandem repeat (TR) of MUC1. Using the SYFPEITHI database, the ideal sequence covering all epitopes of the TR in mouse (Balb/c) and in human (MHC I alleles available) was defined as: GVTSAPDTRPAPGSTAPPAHGVTSA (SEQ ID NO:5).

However, since it is not known whether it is possible to insert in the flagellin a sequence with such a size (25 amino acids) without affecting the structure of the flagella, the following 3 constructs were designed: Fla-MUC1.20 (which include one TR; GVTSAPDTRPAPGSTAPPAH; SEQ ID NO:6), Fla-MUC1-22 (GVTSAPDTRPAPGSTAPPAHGV; SEQ ID NO:7, which includes one TR and 2 amino acid residues that are important in an epitope on the C terminal region of the TR), and the Fla-MUC1.25 above (GVTSAPDTRPAPGSTAPPAHGVTSA; SEQ ID NO:5). The correct conformation of the flagella should allow the Salmonella to rotate and thus can be confirmed under light microscopy.

Experimental Results

Successful generation of a chimeric recombinant flagella with 25 amino acids of MUC1—The Fla-MUC1.25 (comprises SEQ ID NO:22), which includes 25 (SEQ ID NO:5) amino acids of the MUC1 polypeptide, allowed the Salmonella to rotate, indicating that it is possible to insert additional 25 amino acids into the flagellin polypeptide without affecting its structure.

### Example 10

### Effect of Fla-MUC1.25 in Treating Cancer

To test whether the new Fla-MUC1.25 chimeric polypeptide is capable of treating cancer, the recombinant vaccine with the adequate conformation containing the longest insert was used as a vaccine.

Experimental Design

1. Immunization protocol of Fla-MUC1.7+Fla-MUC1.9 or Fla-MUC1.25 in Balb/c mice following implantation of 4T1-MUC1 cells—Female Balb/c mice (8 weeks old) were implanted subcutaneously with 1.5×10⁶ 4T1-MUC1 cells. 10 days post-implantation, mice were immunized as followed:

Group 1 (8 mice): PBS (control group)

Group 2 (10 mice): 50 μg of Fla-MUC1-7+50 μg of Fla-MUC1.9

Group 3 (10 mice): 100 μg of Fla-MUC1.25

Tumor growth was monitored by deducting the tumor size on the first day of immunization to the tumor size on each day. Results are shown in FIG. 16.

2. Immunization protocol of Fla-MUC1.7+Fla-MUC1.9 or Fla-MUC1-25 in human MUC1 transgenic mice following implantation of 4T1-MUC1 cells—Female human MUC1 transgenic (8 weeks old) were implanted subcutaneously with 1.5×10⁶ 4T1-MUC1 cells. 13 days post-implantation, mice were immunized as followed:

Group 1 (8 mice): PBS (control group)

Group 2 (10 mice): 50 μg of Fla-MUC1.7+50 μg of Fla-MUC1.9

Group 3 (10 mice): 100 μg of Fla-MUC1.25

Tumor growth was monitored by deducting the tumor size on the first day of immunization to the tumor size on each day. Results are shown in FIGS. 19 and 20.

Experimental Results

Significant inhibition of tumor growth following immunization with the Fla-MUC1.25 recombinant polypeptide—The effect of Fla-MUC1.25 on tumor growth was tested in comparison to the combination of the recombinant vaccine Fla-MUC1.7 and Fla-MUC1.9. As is clearly shown in FIG. 16, immunization with the Fla-MUC1.25 (which comprises SEQ ID NO:22) resulted in a significant inhibition of tumor growth compared to the two other immunized groups (PBS and the combined Fla-MUC1.7 and Fla-MUC1.9 vaccine).

Significant inhibition of tumor growth in human MUC1 transgenic mice immunized with the Fla-MUC1-25 recombinant polypeptide—In another experiment performed using the same procedure in the human MUC1 transgenic mice model of the F8 generation, similar results were obtained (FIG. 19). Additionally, lung metastasis were monitored (FIG. 20) 54 days post-implantation. The group of mice that were immunized with both preparation (Fla-MUC1.7+Fla-MUC1.9 or Fla-MUC1.25 present twice less metastasis to the lung as compared to the mice immunized with PBS.

Analysis and Discussion

Fla-MUC1.7 and Fla-MUC1.9 (and the combination of both vaccines) display significant efficiency in therapeutic treatment in single injection without adjuvant in slowing down tumor growth in Balb/c and in human MUC1 transgenic mice bearing tumor. Eventually, a second immunization can be beneficial in boosting a second immune response against the tumor. It is of great importance to mention that the lack of need of adjuvant is a non negligible advantage, since new and more efficient adjuvant for human use are still to be engineered (DT O'Hagan et al. 2001).

As prophylactic treatment, the efficiency of Fla-MUC1.7 in slowing down the growth of the tumor in pre-immunized mice is also suggested, although several immunizations with adjuvant seem to be required.

The new recombinant flagellin carrying 25 amino acids of the tandem repeat of MUC1 has been now tested as an anti cancer vaccine, in order to cover all the possible epitopes included in the extracellular domain of MUC1 regardless to the MHC specificity. This recombinant vaccine displayed even better efficiency inhibiting tumor growth.

The mechanism of action of the tested vaccines (the compositions of the present invention) is likely to be related to cellular response (e.g., due to the induction of Th1 response), and will be further investigated.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES

### Additional References are Cited in Text

1. http://www.who.int
2. Levi R, Arnon R. Synthetic recombinant influenza vaccine induces efficient long-term immunity and cross-strain protection. Vaccine. 1996 January; 14(1):85-92.
3. Apostolopoulos V, McKenzei I F. Cellular mucins: targets for immunotherapy. Crit. Rev. Immunol. 14: 293-309, 1994.
4. Finn O J. Cancer vaccines: between the idea and the reality. Nature. 3: 630-641, 2003.
5. Ben-Yedidia T, Tarrab-Hazdai R, Schechtman D, Arnon R. Intranasal administration of synthetic recombinant peptide-based vaccine protects mice from infection by Schistosoma mansoni. Infect Immun. 1999 September; 67(9):4360-6.
6. Ben-Yedidia T, Marcus H, Reisner Y and Arnon R. Intranasal administration of peptide vaccine protects human/mouse radiation chimera from influenza infection. Int. J. Immunol. 11, 1043-4051 (1999).
7. Ben-Yedidia T and Arnon R. Effect of pre-existing carrier immunity on the efficient of synthetic influenza vaccine. 1 mm. Lett. 4:9-15, 1995.
8. Fumitake Hayashi et al. The innate immune response to bacterial flagellin is mediated by Toll-like receptor 5. Nature. 410; 26 Apr. 2001.
9. S. Akira et al. Toll-like receptors: critical proteins linking innate and acquired immunity. Nature Immunology, August 2001, Vol 2 no 8: 675-680.
10. T K Means et al. The Toll-like receptor 5 stimulus bacterial flagellin induces maturation and chemokine production in human dendritic cells. J. Immunology, 2003, 170: 5165-5175.
11. D V Duin et al. Triggering TLR signalling in vaccination. Trends in imm. 11.05, 2005.
12. Moase E H, Qi W, Ishida T et al. Anti-MUC1 immunoliposomal doxorubicin in the treatment of murine models of metastatic breast cancer. Biochim Biophys Acta 1510:43-55, 2001.
13. Rowse G J, Tempero R M, et al. Tolerance and immunity to MUC1 in a human MUC1 transgenic murine model Cancer Res. 58:315-321, 1998.
14. Nussbaum A K, Kuttler C et al. Using the World Wide Web for predicting CTL epitopes. Curr. Opinion 1 mm. 15: 69-74; 2003.
15. Hans-Georg Rammensee, Jutta Bachmann, Niels Nikolaus Emmerich, Oskar Alexander Bachor, Stefan Stevanovic: SYFPEITHI database for MHC ligands and peptide motifs. Immunogenetics (1999) 50: 213-219. access vi a: www.syfpeithi.de
16. Ibrahim G. F. et al. Method for the isolation of highly purified Salmonella flagellin. J. Clin. Microbiol., 22, 1040-1044, 1991.
17. Cuadros C. Et al. Flagellin fusion proteins as adjuvants or vaccines induce specific immune responses. Infection and Immunity, May 2004, p. 2810-2816.
18. Taylor-Papadimitriou et al. MUC1 and immunobiology of cancer. Journal of mammary gland biology and neoplasia, Vol. 7, No. 2, April 2002.
19. Soo Kie Kim et al. Effect of immunological adjuvant combinations on the antibody and T-cell response to vaccination with MUC1-KLH and GD3-KLH conjugates. Vaccine 19, 530-537 2001.
20. D T O'Hagan et al. Recent developments in adjuvants for vaccines against infectious diseases. Biomolecular engineering 18: 69-85, (2001).

What is claimed is:

1. A method of treating cancer in a subject in need thereof, the method comprising administering to the subject a therapeutically effective amount of a composition which comprises a polypeptide, said polypeptide having an amino acid sequence of a flagellin and an amino acid sequence of a mucin 1, said amino acid sequence of said mucin 1 comprises at least a 7 amino acid sequence of SEQ ID NO:6, thereby treating the cancer in the subject.

2. A composition-of-matter comprising a polypeptide, said polypeptide having an amino acid sequence of a flagellin and an amino acid sequence of a mucin 1, said amino acid sequence of said mucin 1 comprises at least a 7 amino acid sequence of SEQ ID NO:6.

3. A bacterial host cell being transformed with a nucleic acid construct encoding a polypeptide having an amino acid sequence of a flagellin and an amino acid sequence of a mucin 1, said amino acid sequence of said mucin 1 comprises at least a 7 amino acid sequence of SEQ ID NO:6.

4. A pharmaceutical composition comprising a therapeutically effective amount of a composition which comprises a polypeptide, said polypeptide having an amino acid sequence of a flagellin and an amino acid sequence of a mucin 1, said amino acid sequence of said mucin 1 comprises at least a 7 amino acid sequence of SEQ ID NO:6, and a pharmaceutically acceptable carrier.

5. The method of claim 1, wherein said amino acid sequence of said flagellin is a contiguous amino acid sequence.

6. The method of claim 5, wherein said amino acid sequence of said mucin 1 is positioned at an N-terminal end of said contiguous amino acid sequence of said flagellin.

7. The method of claim 5, wherein said amino acid sequence of said mucin 1 is positioned at a C-terminal end of said contiguous amino acid sequence of said flagellin.

8. The method of claim 1, wherein said amino acid sequence of said flagellin is a non-contiguous amino acid sequence.

9. The method of claim 8, wherein said amino acid sequence of said mucin 1 is flanked by two amino acid segments of said non-contiguous amino acid sequence of said flagellin.

10. The method of claim 1, wherein cells of the cancer express MUC1.

11. The method of claim 1, wherein said therapeutically effective amount of said composition is selected capable of eliciting a specific immune response against the cancer in the subject.

12. The method of claim 11, wherein said immune response is a cellular immune response.

13. The method of claim 11, wherein said immune response is capable of inhibiting growth of cells of the cancer.

14. The method of claim 1, wherein said amino acid sequence of said mucin 1 is selected from the group consisting of SEQ ID NO:1, 2, 5, 6 and 7.

15. The method claim 1, wherein the cancer affects glandular epithelium.

16. The method of claim 15, wherein the cancer is selected from the group consisting of breast cancer, lung cancer, salivary gland cancer, gastric cancer, pancreatic cancer, bile duct cancer, kidney cancer, ovarian cancer, uterus cancer, testis cancer, prostate cancer and bladder cancer.

17. The method of claim 1, wherein the cancer is a hematological malignancy.

18. The method of claim 17, wherein said hematological malignancy is selected from the group consisting of lymphoma, AML and myeloma.

19. The method of claim 1, wherein the cancer is cancer metastases.

20. The method of claim 1, wherein said flagellin is a salmonella flagellin.

21. The composition-of-matter of claim 2, wherein said amino acid sequence of said flagellin is a contiguous amino acid sequence.

22. The composition-of-matter of claim 21, wherein said amino acid sequence of said mucin 1 is positioned at an N-terminal end of said contiguous amino acid sequence of said flagellin.

23. The composition-of-matter of claim 21, wherein said amino acid sequence of said mucin 1 is positioned at a C-terminal end of said contiguous amino acid sequence of said flagellin.

24. The composition-of-matter of claim 2, wherein said amino acid sequence of said flagellin is a non-contiguous amino acid sequence.

25. The composition-of-matter of claim 24, wherein said amino acid sequence of said mucin 1 is flanked by two amino acid segments of said non-contiguous amino acid sequence of said flagellin.

26. The composition-of-matter of claim 2, wherein said amino acid sequence of said mucin 1 is selected from the group consisting of SEQ ID NO:1, 2, 5, 6 and 7.

27. The composition-of-matter of claim 2, wherein said flagellin is a salmonella flagellin.


SEQUENCE LISTING (36 sequences)

SEQ ID NO: 1 - protein, 7 aa, Artificial sequence
  MUC1.7 epitope
APDTRPA

SEQ ID NO: 2 - protein, 9 aa, Artificial sequence
  MUC1.9 epitope
RPAPGSTAP

SEQ ID NO: 3 - DNA, 24 bp, Artificial sequence
  Single strand DNA oligonucleotide
gctccggatacccgtccggctgat

SEQ ID NO: 4 - DNA, 30 bp, Artificial sequence
  Single strand DNA oligonucleotide
agaccggctccgggtagcaccgctccggat

SEQ ID NO: 5 - protein, 25 aa, Artificial sequence
  MUC1.25 epitope
GVTSAPDTRPAPGSTAPPAHGVTSA

SEQ ID NO: 6 - protein, 20 aa, Artificial sequence
  MUC-1 repeated sequence
GVTSAPDTRPAPGSTAPPAH

SEQ ID NO: 7 - protein, 22 aa, Artificial sequence
  MUC1.22 epitope
GVTSAPDTRPAPGSTAPPAHGV

SEQ ID NO: 8 - protein, 251 aa, Artificial sequence
  Fla-MUC1.7 chimeric polypeptide
RVRELAVQSANGTNSQSDLDSIQAEITQRLNEIDRVSGQTQFNGVKVLAQDNTLTIQVGANDGETIDIDLKEISSKTLGLDKLNVQDAYTPKETAVTVDKTTYKNGTDTITAQSNTDAPDTRPADIKFKDGQYYLDVKGGASAGVYKATYDETTKKVNIDTTDKTPLATAEATAIRGTATITHNQIAEVTKEGVDTTTVAAQLAAAGVTGADKDNTSLVKLSFEDKNGKVIDGGYAVKMGDDFYAATYDEK

SEQ ID NO: 9 - protein, 253 aa, Artificial sequence
  Fla-MUC1.9 chimeric polypeptide
RVRELAVQSANGTNSQSDLDSIQAEITQRLNEIDRVSGQTQFNGVKVLAQDNTLTIQVGANDGETIDIDLKEISSKTLGLDKLNVQDAYTPKETAVTVDKTTYKNGTDTITAQSNTDRPAPGSTAPDIKFKDGQYYLDVKGGASAGVYKATYDETTKKVNIDTTDKTPLATAEATAIRGTATITHNQIAEVTKEGVDTTTVAAQLAAAGVTGADKDNTSLVKLSFEDKNGKVIDGGYAVKMGDDFYAATYDEK

SEQ ID NO: 10 - protein, 243 aa, Artificial sequence
  Salmonella munchen derived recombinant Flagellin
RVRELAVQSANGTNSQSDLDSIQAEITQRLNEIDRVSGQTQFNGVKVLAQDNTLTIQVGANDGETIDIDLKEISSKTLGLDKLNVQDAYTPKETAVTVDKTTYKNGTDTITAQSNTDIKFKDGQYYLDVKGGASAGVYKATYDETTKKVNIDTTDKTPLATAEATAIRGTATITHNQIAEVTKEGVDTTTVAAQLAAAGVTGADKDNTSLVKLSFEDKNGKVIDGGYAVKMGDDFYAATYDEK

SEQ ID NO: 11 - protein, 509 aa, Salmonella muenchen
KEKIMAQVINTNSLSLLTQNNLNKSQSALGTAIERLSSGLRINSAKDDAAGQAIANRFTANIKGLTQASRNANDGISIAQTTEGALNEINNNLQRVRELAVQSANGTNSQSDLDSIQAEITQRLNEIDRVSGQTQFNGVKVLAQDNTLTIQVGANDGETIDIDLKEISSKTLGLDKLNVQDAYTPKETAVTVDKTTYKNGTDTITAQSNTDIQTAIGGGATGVTGADIKFKDGQYYLDVKGGASAGVYKATYDETTKKVNIDTTDKTPLATAEATAIRGTATITHNQIAEVTKEGVDTTTVAAQLAAAGVTGADKDNTSLVKLSFEDKNGKVIDGGYAVKMGDDFYAATYDEKQVQLLLNNHYTDGAGVLQTGAVKFGGANGKSEVVTATVGKTYLASDLDKHNFRTGGELKEVNTDKTENPLQKIDAALAQVDTLRSDLGAVQNRFNSAITNLGNTVNNLSSARSRIEDSDYATEVSNMSRAQILQQAGTSVLAQANQVPQNVLSLLR

SEQ ID NO: 12 - protein, 1255 aa, Homo sapiens
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSSVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAAASANL

SEQ ID NO: 13 - protein, 9 aa, Artificial sequence
  Synthetic peptide
AHGVTSAPD

SEQ ID NO: 14 - protein, 9 aa, Artificial sequence
  Synthetic peptide
STAPPAHGV

SEQ ID NO: 15 - protein, 516 aa, Artificial sequence
  MUC1.7-Fla N′ chimeric polypeptide
APDTRPAKEKIMAQVINTNSLSLLTQNNLNKSQSALGTAIERLSSGLRINSAKDDAAGQAIANRFTANIKGLTQASRNANDGISIAQTTEGALNEINNNLQRVRELAVQSANGTNSQSDLDSIQAEITQRLNEIDRVSGQTQFNGVKVLAQDNTLTIQVGANDGETIDIDLKEISSKTLGLDKLNVQDAYTPKETAVTVDKTTYKNGTDTITAQSNTDIQTAIGGGATGVTGADIKFKDGQYYLDVKGGASAGVYKATYDETTKKVNIDTTDKTPLATAEATAIRGTATITHNQIAEVTKEGVDTTTVAAQLAAAGVTGADKDNTSLVKLSFEDKNGKVIDGGYAVKMGDDFYAATYDEKQVQLLLNNHYTDGAGVLQTGAVKFGGANGKSEVVTATVGKTYLASDLDKHNFRTGGELKEVNTDKTENPLQKIDAALAQVDTLRSDLGAVQNRFNSAITNLGNTVNNLSSARSRIEDSDYATEVSNMSRAQILQQAGTSVLAQANQVPQNVLSLLR

SEQ ID NO: 16 - protein, 516 aa, Artificial sequence
  MUC1.7-Fla C′ chimeric polypeptide
KEKIMAQVINTNSLSLLTQNNLNKSQSALGTAIERLSSGLRINSAKDDAAGQAIANRFTANIKGLTQASRNANDGISIAQTTEGALNEINNNLQRVRELAVQSANGTNSQSDLDSIQAEITQRLNEIDRVSGQTQFNGVKVLAQDNTLTIQVGANDGETIDIDLKEISSKTLGLDKLNVQDAYTPKETAVTVDKTTYKNGTDTITAQSNTDIQTAIGGGATGVTGADIKFKDGQYYLDVKGGASAGVYKATYDETTKKVNIDTTDKTPLATAEATAIRGTATITHNQIAEVTKEGVDTTTVAAQLAAAGVTGADKDNTSLVKLSFEDKNGKVIDGGYAVKMGDDFYAATYDEKQVQLLLNNHYTDGAGVLQTGAVKFGGANGKSEVVTATVGKTYLASDLDKHNFRTGGELKEVNTDKTENPLQKIDAALAQVDTLRSDLGAVQNRFNSAITNLGNTVNNLSSARSRIEDSDYATEVSNMSRAQILQQAGTSVLAQANQVPQNVLSLLRAPDTRPA

SEQ ID NO: 17 - DNA, 729 bp, Artificial sequence
  Salmonella munchen derived recombinant Flagellin coding sequence
cgtgtgcgtgaactggcggttcagtctgctaacggtactaactcccagtctgaccttgactctatccaggctgaaatcacccagcgtctgaacgaaatcgaccgtgtatccggtcagactcagttcaacggcgtgaaagtcctggcgcaggacaacaccctgaccatccaggttggtgccaacgacggtgaaactattgatattgatttaaaagaaattagctctaaaacactgggacttgataagcttaatgtccaggatgcctacaccccgaaagaaactgctgtaaccgttgataaaactacctataaaaatggtacagatactattacagcccagagcaatactgatatcaaatttaaagatggtcaatactatttagatgttaaaggcggtgcttctgctggtgtttataaagccacttatgatgaaactacaaagaaagttaatattgatacgactgataaaactccgttagcaactgcggaagctacagctattcggggaacggccactataacccacaaccaaattgctgaagtaacaaaagagggtgttgatacgaccacagttgcggctcaacttgctgctgcaggggttactggtgccgataaggacaatactagccttgtaaaactatcgtttgaggataaaaacggtaaggttattgatggtggctatgcagtgaaaatgggcgacgatttctatgccgctacatatgatgagaaa

SEQ ID NO: 18 - DNA, 753 bp, Artificial sequence
  Fla-MUC1.7 chimeric polypeptide coding sequence
cgtgtgcgtgaactggcggttcagtctgctaacggtactaactcccagtctgaccttgactctatccaggctgaaatcacccagcgtctgaacgaaatcgaccgtgtatccggtcagactcagttcaacggcgtgaaagtcctggcgcaggacaacaccctgaccatccaggttggtgccaacgacggtgaaactattgatattgatttaaaagaaattagctctaaaacactgggacttgataagcttaatgtccaggatgcctacaccccgaaagaaactgctgtaaccgttgataaaactacctataaaaatggtacagatactattacagcccagagcaatactgatgctccggatacccgtccggctgatatcaaatttaaagatggtcaatactatttagatgttaaaggcggtgcttctgctggtgtttataaagccacttatgatgaaactacaaagaaagttaatattgatacgactgataaaactccgttagcaactgcggaagctacagctattcggggaacggccactataacccacaaccaaattgctgaagtaacaaaagagggtgttgatacgaccacagttgcggctcaacttgctgctgcaggggttactggtgccgataaggacaatactagccttgtaaaactatcgtttgaggataaaaacggtaaggttattgatggtggctatgcagtgaaaatgggcgacgatttctatgccgctacatatgatgagaaa

SEQ ID NO: 19 - DNA, 759 bp, Artificial sequence
  Fla-MUC1.9 chimeric polypeptide coding sequence
cgtgtgcgtgaactggcggttcagtctgctaacggtactaactcccagtctgaccttgactctatccaggctgaaatcacccagcgtctgaacgaaatcgaccgtgtatccggtcagactcagttcaacggcgtgaaagtcctggcgcaggacaacaccctgaccatccaggttggtgccaacgacggtgaaactattgatattgatttaaaagaaattagctctaaaacactgggacttgataagcttaatgtccaggatgcctacaccccgaaagaaactgctgtaaccgttgataaaactacctataaaaatggtacagatactattacagcccagagcaatactgatagaccggctccgggtagcaccgctccggatatcaaatttaaagatggtcaatactatttagatgttaaaggcggtgcttctgctggtgtttataaagccacttatgatgaaactacaaagaaagttaatattgatacgactgataaaactccgttagcaactgcggaagctacagctattcggggaacggccactataacccacaaccaaattgctgaagtaacaaaagagggtgttgatacgaccacagttgcggctcaacttgctgctgcaggggttactggtgccgataaggacaatactagccttgtaaaactatcgtttgaggataaaaacggtaaggttattgatggtggctatgcagtgaaaatgggcgacgatttctatgccgctacatatgatgagaaa

SEQ ID NO: 20 - protein, 256 aa, Artificial sequence
  Fla-NP chimeric polypeptide
RVRELAVQSANGTNSQSDLDSIQAEITQRLNEIDRVSGQTQFNGVKVLAQDNTLTIQVGANDGETIDIDLKEISSKTLGLDKLNVQDAYTPKETAVTVDKTTYKNGTDTITAQSNTDTYQRTRALVRTGDIKFKDGQYYLDVKGGASAGVYKATYDETTKKVNIDTTDKTPLATAEATAIRGTATITHNQIAEVTKEGVDTTTVAAQLAAAGVTGADKDNTSLVKLSFEDKNGKVIDGGYAVKMGDDFYAATYDEK

SEQ ID NO: 21 - protein, 12 aa, Artificial sequence
  A polypeptide derived from the nucleoprotein of influenza NP 147-158
TYQRTRALVRTG

SEQ ID NO: 22 - protein, 259 aa, Artificial sequence
  Fla-MUC1.25 chimeric polypeptide
RVRELAVQSANGTNSQSDLDSIQAEITQRLNEIDRVSGQTQFNGVKVLAQDNTLTIQVGANDGETIDIDLKEISSKTLGLDKLNVQDAYTPKETAVTVDKTTYKTGTDGVTSAPDTRPAPGSTAPPAHGVTSAIKFKDGQYYLDVKGGASAGVYKATYDETTKKVNIDTTDKTPLATAEATAIRGTATITHNQIAEVTKEGVDTTTVAAQLAAAGVTGADKDNTSLVKLSFEDKNGKVIDGGYAVKMGDDFYAATYDEK

SEQ ID NO: 23 - DNA, 60 bp, Artificial sequence
  Single strand DNA oligonucleotide
ggcgtgacctcggcgccggatacccgcccggcgccgggctcgaccgcgccgccggcgcat

SEQ ID NO: 24 - DNA, 66 bp, Artificial sequence
  Single strand DNA oligonucleotide
ggcgtgacctcggcgccggatacccgcccggcgccgggctcgaccgcgccgccggcgcatggcgtg

SEQ ID NO: 25 - DNA, 75 bp, Artificial sequence
  Single strand DNA oligonucleotide
ggcgtgacctcggcgccggatacccgcccggcgccgggctcgaccgcgccgccggcgcatggcgtgacctcggcg

SEQ ID NO: 26 - protein, 254 aa, Artificial sequence
  Fla-MUC1.20 chimeric polypeptide
RVRELAVQSANGTNSQSDLDSIQAEITQRLNEIDRVSGQTQFNGVKVLAQDNTLTIQVGANDGETIDIDLKEISSKTLGLDKLNVQDAYTPKETAVTVDKTTYKTGTDGVTSAPDTRPAPGSTAPPAHIKFKDGQYYLDVKGGASAGVYKATYDETTKKVNIDTTDKTPLATAEATAIRGTATITHNQIAEVTKEGVDTTTVAAQLAAAGVTGADKDNTSLVKLSFEDKNGKVIDGGYAVKMGDDFYAATYDEK

SEQ ID NO: 27 - protein, 256 aa, Artificial sequence
  Fla-MUC1.22 chimeric polypeptide
RVRELAVQSANGTNSQSDLDSIQAEITQRLNEIDRVSGQTQFNGVKVLAQDNTLTIQVGANDGETIDIDLKEISSKTLGLDKLNVQDAYTPKETAVTVDKTTYKTGTDGVTSAPDTRPAPGSTAPPAHGVIKFKDGQYYLDVKGGASAGVYKATYDETTKKVNIDTTDKTPLATAEATAIRGTATITHNQIAEVTKEGVDTTTVAAQLAAAGVTGADKDNTSLVKLSFEDKNGKVIDGGYAVKMGDDFYAATYDEK

SEQ ID NO: 28 - DNA, 729 bp, Artificial sequence
  Salmonella munchen derived recombinant Flagellin coding sequence
cgtgtgcgtgaactggcggttcagtctgctaacggtactaactcccagtctgaccttgactctatccaggctgaaatcacccagcgtctgaacgaaatcgaccgtgtatccggtcagactcagttcaacggcgtgaaagtcctggcgcaggacaacaccctgaccatccaggttggtgccaacgacggtgaaactattgatattgatttaaaagaaattagctctaaaacactgggacttgataagcttaatgtccaggatgcctacaccccgaaagaaactgctgtaaccgttgataaaactacctataaaaccggtacagatactattacagcccagagcaatactgatatcaaatttaaagatggtcaatactatttagatgttaaaggcggtgcttctgctggtgtttataaagccacttatgatgaaactacaaagaaagttaatattgatacgactgataaaactccgttagcaactgcggaagctacagctattcggggaacggccactataacccacaaccaaattgctgaagtaacaaaagagggtgttgatacgaccacagttgcggctcaacttgctgctgcaggggttactggtgccgataaggacaatactagccttgtaaaactatcgtttgaggataaaaacggtaaggttattgatggtggctatgcagtgaaaatgggcgacgatttctatgccgctacatatgatgagaaa

SEQ ID NO: 29 - DNA, 86 bp, Artificial sequence
  Single strand DNA oligonucleotide
ccggtacagatggcgtgacctcggcgccggatacccgcccggcgccgggctcgaccgcgccgccggcgcatggcgtgacctcggcg

SEQ ID NO: 30 - DNA, 82 bp, Artificial sequence
  Single strand DNA oligonucleotide
cgccgaggtcacgccatgcgccggcggcgcggtcgagcccggcgccgggcgggtatccggcgccgaggtcacgccatctgta

SEQ ID NO: 31 - protein, 243 aa, Artificial sequence
  Salmonella munchen derived recombinant Flagellin
RVRELAVQSANGTNSQSDLDSIQAEITQRLNEIDRVSGQTQFNGVKVLAQDNTLTIQVGANDGETIDIDLKEISSKTLGLDKLNVQDAYTPKETAVTVDKTTYKTGTDTITAQSNTDIKFKDGQYYLDVKGGASAGVYKATYDETTKKVNIDTTDKTPLATAEATAIRGTATITHNQIAEVTKEGVDTTTVAAQLAAAGVTGADKDNTSLVKLSFEDKNGKVIDGGYAVKMGDDFYAATYDEK

SEQ ID NO: 32 - DNA, 1530 bp, Salmonella muenchen
aaggaaaagatcatggcacaagtcattaatacaaacagcctgtcgctgttgacccagaataacctgaacaaatcccagtccgctctgggcaccgctatcgagcgtctgtcttccggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacggtactaactcccagtctgaccttgactctatccaggctgaaatcacccagcgtctgaacgaaatcgaccgtgtatccggtcagactcagttcaacggcgtgaaagtcctggcgcaggacaacaccctgaccatccaggttggtgccaacgacggtgaaactattgatattgatttaaaagaaattagctctaaaacactgggacttgataagcttaatgtccaggatgcctacaccccgaaagaaactgctgtaaccgttgataaaactacctataaaaatggtacagatactattacagcccagagcaatactgatatccaaactgcaattggcggtggtgcaacgggggttactggggctgatatcaaatttaaagatggtcaatactatttagatgttaaaggcggtgcttctgctggtgtttataaagccacttatgatgaaactacaaagaaagttaatattgatacgactgataaaactccgttagcaactgcggaagctacagctattcggggaacggccactataacccacaaccaaattgctgaagtaacaaaagagggtgttgatacgaccacagttgcggctcaacttgctgctgcaggggttactggtgccgataaggacaatactagccttgtaaaactatcgtttgaggataaaaacggtaaggttattgatggtggctatgcagtgaaaatgggcgacgatttctatgccgctacatatgatgagaaacaggtacaattactgctaaacaaccactatacagatggtgctggcgtgctccaaactggagctgtgaaatttggtggcgcaaatggtaaatctgaagttgttactgctaccgtaggtaaaacttacttagcaagcgaccttgacaaacataacttcagaacaggcggtgagcttaaagaggttaatacagataagactgaaaacccactgcagaaaattgatgctgccttggcacaggttgatacacttcgttctgacctgggtgcggtacagaaccgtttcaactccgctatcaccaacctgggcaataccgtaaataacctgtcttctgcccgtagccgtatcgaagattccgactacgcgaccgaagtctccaacatgtctcgcgcgcagattctgcagcaggccggtacctccgttctggcgcaggctaaccaggttccgcaaaacgtcctctctttactgcgttaa

SEQ ID NO: 33 - DNA, 71 bp, Artificial sequence
  Single strand DNA oligonucleotide
ccggtacagatggcgtgacctcggcgccggatacccgcccggcgccgggctcgaccgcgccgccggcgcat

SEQ ID NO: 34 - DNA, 67 bp, Artificial sequence
  Single strand DNA oligonucleotide
atgtctaccgcactggagccgcggcctatgggcgggccgcggcccgagctggcgcggcggccgcgta

SEQ ID NO: 35 - DNA, 77 bp, Artificial sequence
  Single strand DNA oligonucleotide
ccggtacagatggcgtgacctcggcgccggatacccgcccggcgccgggctcgaccgcgccgccggcgcatggcgtg

SEQ ID NO: 36 - DNA, 73 bp, Artificial sequence
  Single strand DNA oligonucleotide
atgtctaccgcactggagccgcggcctatgggcgggccgcggcccgagctggcgcggcggccgcgtaccgcac